(19) United States
(12) Patent Application Publication
(10) Pub. No.: US 2024/0342106 A1
(43) Pub. Date: Oct. 17, 2024

(54) NANOPARTICLES AND USES THEREOF FOR TREATMENT OF HUMAN IMMUNODEFICIENCY VIRUS

(71) Applicants: UNIVERSITY OF MIAMI, Miami, FL (US); THE FLORIDA INTERNATIONAL UNIVERSITY BOARD OF TRUSTEES, Miami, FL (US)

(72) Inventors: Shanta DHAR, Miami, FL (US); Nagesh KOLISHETTI, Miami, FL (US); Bapurao SURNAR, Miami, FL (US); Madhavan NAIR, Miami, FL (US); Anuj SHAH, Miami, FL (US); Michal TOBOREK, Miami, FL (US)

(21) Appl. No.: 18/292,258

(22) PCT Filed: Jul. 27, 2022
(86) PCT No.: PCT/US2022/074226

§ 371 Date: Jan. 25, 2024

Related U.S. Application Data
(60) Provisional application No. 63/226,034, filed on Jul. 27, 2021.

(51) Int. Cl. A61K 9/51; A61K 31/122; A61K 31/403; A61K 31/427; A61K 31/435; A61K 31/439; A61K 31/4418; A61K 31/4433; A61K 31/4439; A61K 31/47; A61K 31/4709; A61K 31/4741; A61K 31/496; A61K 31/497; A61K 31/4985; A61K 31/499; A61K 31/5025; A61K 31/505; A61K 31/506; A61K 31/513; A61K 31/519; A61K 31/52; A61K 31/522; A61K 31/536; A61K 31/5365; A61K 31/537; A61K 31/551; A61K 31/573; A61K 31/616; A61K 31/635; A61K 31/675; A61K 31/69; A61K 31/7072; A61K 38/16; A61K 38/18; A61K 39/395; A61K 47/54; A61P 31/18

(52) U.S. Cl. CPC A61K 9/5153; A61K 31/122; A61K 31/403; A61K 31/427; A61K 31/435; A61K 31/439; A61K 31/4418; A61K 31/4433; A61K 31/4439; A61K 31/47; A61K 31/4709; A61K 31/4741; A61K 31/496; A61K 31/497; A61K 31/4985; A61K 31/499; A61K 31/5025; A61K 31/505; A61K 31/506; A61K 31/513; A61K 31/519; A61K 31/52; A61K 31/522; A61K 31/536; A61K 31/5365; A61K 31/537; A61K 31/551; A61K 31/573; A61K 31/616; A61K 31/635; A61K 31/675; A61K 31/69; A61K 31/7072; A61K 38/16; A61K 38/18; A61K 39/3955; A61K 47/548; A61P 31/18

(57) ABSTRACT

The present disclosure relates to nanoparticle compositions and uses thereof for treating or preventing HIV infection.

### CROSS-REFERENCE TO RELATED APPLICATIONS

This application claims the benefit of U.S. Provisional Application No. 63/226,034, filed Jul. 27, 2021, which is expressly incorporated herein by reference in its entirety.

### STATEMENT REGARDING FEDERALLY SPONSORED RESEARCH

This invention was made with government support under Grant Nos. DA037838 and DA044579 awarded by the National Institutes of Health. The government has certain rights in the invention.

### FIELD

The present disclosure relates to compositions and uses thereof for treating and/or preventing human immunodeficiency virus (HIV) infection.

### BACKGROUND

Combination treatment regimens of potent antiretroviral drugs can reduce human immunodeficiency virus (HIV) to undetectable levels in the blood. However, no current therapies can tackle the virus reservoirs in central memory CD4 T-cells, hematopoietic stem cells, dendritic cells as well as viral reservoirs that accumulate in the brain. The selective permeability of the blood-brain barrier (BBB) poses a challenge to delivering enough drugs into the brain, leading to HIV's persistence as a formidable chronic illness to treat. What is needed are novel compositions and methods for treating HIV infection in central neural system. Pregnant women have limited treatment options as most small molecule based lipophilic drugs cross the placental barrier and cause harm to the fetus. Thus, therapeutic technologies which do not cross the placental barrier are in immediate need.

### SUMMARY

Disclosed herein are nanoparticles and methods of use thereof. In some aspect, disclosed herein is a method of reducing a level of human immunodeficiency virus (HIV) in a pregnant subject in need thereof, comprising administering to the pregnant subject a therapeutically effective amount of the nanoparticle, wherein the nanoparticle comprises:
a mitochondrial targeting moiety;
an anti-HIV therapeutic agent; and/or
one or more of an anti-oxidant agent and/or an anti-inflammatory agent.

In some embodiments, the anti-HIV therapeutic agent and the one or more of an anti-oxidant agent and/or an anti-inflammatory agent can be found within one nanoparticle. In some embodiments, the anti-HIV therapeutic agent and the one or more of an anti-oxidant agent and/or an anti-inflammatory agent can be found within separate nanoparticles (for example, the composition comprises nanoparticles loaded with an anti-HIV therapeutic agent, and separate nanoparticles loaded with an anti-oxidant agent and/or nanoparticles loaded with an anti-inflammatory agent).

In some embodiments, the nanoparticle comprises the anti-oxidant agent or the anti-inflammatory agent. In some embodiments, the anti-oxidant comprises Coenzyme Q10 (CoQ₁₀). In some embodiments, the anti-inflammatory agent comprises a prodrug of aspirin or a prodrug of prednisone. In some embodiments, the prodrug of aspirin is Oc-G2-(Asp)₄ (Asp4). In some embodiments, the prodrug of prednisone is Oc-[G-2]-(Pred)₄.

In some embodiments, the mitochondrial targeting moiety comprises a triphenyl phosphonium (TPP) moiety or a derivative thereof.

In some embodiments, the anti-HIV therapeutic agent comprises a protease inhibitor, an integrase inhibitor, a fusion inhibitor, a nucleoside reverse transcriptase inhibitor (NRTI), a nucleotide reverse transcriptase inhibitor (NtRTI), a non-nucleoside reverse-transcriptase inhibitor (NNRTI), a CCR5 antagonist, a post attachment inhibitor, or a maturation inhibitor.

In some embodiments, the integrase inhibitor comprises raltegravir, elvitegravir, dolutegravir, bictegravir, V-165, BI 224436, MK-2048, Lens epithelial derived growth factor LEDGF, or cabotegravir.

In some embodiments, the protease inhibitor comprises amprenavir, lopinavir Kaletra, saquinavir, indinavir, ritonavir, nelfinavir, atazanavir, fosamprenavir, tipranavir, darunavir, DMP450, PNU-140690, ABT-378, PD178390, asunaprevir, boceprevir, grazoprevir, glecaprevir, paritaprevir, simeprevir, telaprevir, tanomastat, batimastat, or bortezomib.

In some embodiments, the NRTI comprises zidovudine, didanosine, didanosine EC, zalcitabine, stavudine, lamivudine, abacavir, emtricitabine, entecavir, or Truvada.

In some embodiments, the NtRTI comprises tenofovir or adefovir.

In some embodiments, the NNRTI comprises efavirenz, nevirapine, delavirdine, etravirine, rilpivirine, or doravirine.

In some embodiments, the CCR5 antagonist comprises leronlimab, aplaviroc, vicriviroc, maraviroc, or INCB009471.

In some embodiments, the fusion inhibitor comprises efuvirtide.

In some embodiments, the anti-HIV therapeutic agent comprises saquinavir, darunavir, elvitegravir, dolutegravir, raltegravir, bictegravir, efavirenz, delavirdine, or stavudine.

In some embodiments, the anti-HIV therapeutic agent comprises saquinavir, efavirenz, or elvitegravir.

In some embodiments, the nanoparticle comprises poly(lactic-co-glycolic acid) (PLGA)-block(b)-polyethylene glycol (PEG).

In some embodiments, the nanoparticle has a diameter of about 100 nm or less.

In some embodiments, the nanoparticle crosses a blood brain barrier. In some embodiments, the nanoparticle accumulates in the brain. In some embodiments, the nanoparticle does not cross a placental barrier. In some embodiments, the administration of the nanoparticle reduces a level of HIV in the central nervous system of the pregnant subject. In some embodiments, the pregnant subject has HIV-associated neurocognitive disorder (HAND).

In some aspects, disclosed herein is a method for treating human immunodeficiency virus (HIV)-associated neurocognitive disorder (HAND) in a subject in need thereof, comprising administering a therapeutically effective amount of the nanoparticle of any preceding aspect.

In some aspects, disclosed herein is a method for reducing a level of human immunodeficiency virus (HIV) in the central nervous system of a pregnant subject in need thereof, comprising administering to the pregnant subject a therapeutically effective amount of the nanoparticle of any one of preceding aspect.

### BRIEF DESCRIPTION OF DRAWINGS

The accompanying figures, which are incorporated in and constitute a part of this specification, illustrate aspects described below.

FIG. 1 shows BBB-penetrating T-NPs containing ARTs, antioxidant CoQ10, and a prodrug of aspirin for improvement of mitochondrial functions in astrocytes and reduction of inflammation in microglia to promote neuronal protection in HIV-infected brain under drug abuse condition.

FIGS. 2A-2D. FIG. 2A shows structures of ARTs investigated for inclusion in the core of targeted NPs, along with their classification based on molecular target: protease inhibitor (PI), integrase inhibitor (II), and non-nucleoside reverse transcriptase inhibitor (NNRTI). FIG. 2B shows heat maps for diameter, zeta potential, percent loading, and percent encapsulation efficiency of ART-loaded NPs. FIG. 2C shows TEM images of T-EFV-NP, T-EVG-NP, and T-DRV-NP. FIG. 2D shows release profiles of EFV, EVG, and DRV from T-NPs at pH 7.4 at 37° C.

FIGS. 3A-3D. FIG. 3A shows elevated intracellular ROS levels in microglia cells treated with SQV, EFV, DRV, DTG, EVG or the nano-formulations T-SQV-NP, T-EFV-NP, T-DRV-NP, T-DTG-NP, T-EVG-NP at a concentration of 1.0 μM for 24 h. FIG. 3B shows inflammatory responses by the microglia cells by measuring the excretion of IL-1β in the media when these cells were treated with SQV, EFV, DRV, DTG, EVG or the nano-formulations T-SQV-NP, T-EFV-NP, T-DRV-NP, T-DTG-NP, T-EVG-NP at a concentration of 1.0 μM for 24 h production in microglia cells. Effects of (FIG. 3C) cocaine and (FIG. 3D) meth on mitochondrial basal respiration, maximal respiration, and ATP production as determined by Seahorse MitoStress analyses in microglia cells. The abuse drug concentration was varied from 0 to 500 μM and cells were treated for 24 h.

FIGS. 4A-4D. Better distribution of intravenously injected T-ARV-NPs in the brain and blood of female Balb/c mice for (FIG. 4A) EFV and T-EFV-NP, (FIG. 4B) EVG and T-EVG-NP and (FIG. 4C) DRV and T-DRV-NP. FIG. 4D shows that alanine aminotransferase (ALT) and aspartate aminotransferase (AST) levels in the blood plasma. The animals were dosed with 40 mg/kg with respect to the ARVs.

FIGS. 5A-5D. FIG. 5A shows a timeline showing the experimental details of HIV-induced generation of inflammatory and ROS responses in microglia is reversed by T-(Asp)₄-NP and T-CoQ10-NP formulations. HIV-infected human microglia cells were treated with drugs of abuse meth to elevate the responses further. The cells were then treated with T-EVG-NP or T-EFV-NP and then treated with T-(Asp)4-NP and T-CoQ10-NP. Inflammatory responses by analyzing IL-6 and TNF-α (FIG. 5B) in presence of T-EFV-NP and (FIG. 5C) in presence of T-EVG-NP. FIG. 5D shows reversal of mitochondrial oxidative stress by Mito-SOX assay from the experiment detailed in (FIG. 5A). Scale bar=50 μm. For IL-6: ***=0.0003; **=0.0022; *=0.0252. For TNF-α: ***=0.0005; **=0.0039; *=0.0158.

FIGS. 6A-6D. FIG. 6A shows a schematic of the experimental approach where microglia cells were fluorescently labeled with mito-GFP, followed by treatment with TAT peptide, methamphetamine and a combination of T-DRV/EFV/ELV-NP. These microglia cells were then co-cultured with astrocytes treated with a combination of T-(Asp)4-NP+T-CoQ10-NP. FIG. 6B shows cellular levels of the inflammatory markers IL-1β, IL-6, and TNF-α in microglia after co-culturing with NP-treated astrocytes. FIGS. 6C-6D show (FIG. 6C) ATP levels and (FIG. 6D) ROS levels in microglia after nourishing with astrocytes. AST: Human astrocyte; AST*: Neuroprotectant-loaded NP treated human astrocytes.

FIG. 7 shows determination of mitochondrial ROS using in fluorescently labeled microglia cells which were treated with TAT peptide, methamphetamine, and a combination of T-DRV/EFV/ELV-NP, followed by co-culture with astrocytes which were treated with a combination of T-(Asp)4-NP+T-CoQ10-NP (scale bar=50 μm).

FIGS. 8A-8E show efficacy studies in EcoHIV infected Meth treated animal model. C57BL/6 male mice were divided into 12 groups and were assigned to the following treatment groups: control virus+Meth+saline (12 animals); control virus+saline (12 animals); control virus+Meth+T-CoQ₁₀-NP/T-(Asp)₄-NP (13 animals); EcoHIV+Meth+saline (11 animals); EcoHIV+Meth+T-CoQ₁₀-NP/T-(Asp)₄-NP (10 animals); EcoHIV+Meth+T-ART-NPs (12 animals); EcoHIV+Meth+T-ART-NP+T-CoQ₁₀-NP/T-(Asp)₄-NP (16 animals); control Virus+Meth+T-CoQ₁₀/(Asp)₄-NPs (11 animals); control Virus+T-ART-NPs (13 animals); control Virus+T-ART-NPs+T-CoQ₁₀/(Asp)₄-NPs (12 animals), saline (3 animals), and EcoHIV+saline (3 animals). FIG. 8A shows timeline illustrating infection of C57BL/6 male mice with either control virus (pBMN-I-GFP) or chimeric HIV-NDK (EcoHIV, 1 μg of p24), followed by one-week multiday exposure to Meth (0.2 mg/kg at each injection for 5 days) and subsequent two-week treatment with T-ART-NPs (5 mg/kg with respect to the drug) and T-CoQ₁₀/(Asp)₄-NPs (20 mg/kg with respect to the drug). FIG. 8B shows levels of EcoHIV p24 antigen in blood by ELISA and in the brain by RT-PCR. FIG. 8C shows inflammatory markers IL-1β and TNF-α as measured by ELISA in blood plasma. FIG. 8D shows neuroinflammation markers C3 and OLFM1 measured by RT-PCR in the brain. FIG. 8E shows ROS markers, glutamate-cysteine ligase catalytic subunit (GCLC), glutamate-cysteine ligase modifier subunit (GCLM), and glutathione peroxidase 7 (GPX7) measured using RT-PCR in the brain.

FIGS. 9A-9B. FIG. 9A shows ROS levels in isolated astrocytes and neurons from treated mice brain. FIG. 9B shows H&E images of all the tissue sections after the treatment (Scale bar=50 μm). C57BL/6 male mice were divided into 12 groups and were assigned to the following treatment groups: control virus+Meth+saline (12 animals); control virus+saline (12 animals); control virus+Meth+T-CoQ₁₀-NP/T-(Asp)₄-NP (13 animals); EcoHIV+Meth+saline (11 animals); EcoHIV+Meth+T-CoQ₁₀-NP/T-(Asp)₄-NP (10 animals); EcoHIV+Meth+T-ART-NPs (12 animals); EcoHIV+Meth+T-ART-NP+T-CoQ₁₀-NP/T-(Asp)₄-NP (16 animals); control Virus+Meth+T-CoQ₁₀/(Asp)₄-NPs (11 animals); control Virus+T-ART-NPs (13 animals); control Virus+T-ART-NPs+T-CoQ₁₀/(Asp)₄-NPs (12 animals), saline (3 animals), and EcoHIV+saline (3 animals). The mice were treated with either control virus (pBMN-I-GFP) or chimeric HIV-NDK (EcoHIV, 1 μg of p24), followed by one-week multiday exposure to Meth (0.2 mg/kg at each injection for 5 days) and subsequent two-week treatment with T-ART-NPs (5 mg/kg with respect to the drug) and T-CoQ₁₀/(Asp)₄-NPs (20 mg/kg with respect to the drug) via intravenous route.

FIGS. 10A-10D. FIG. 10A shows schematic representation of in vitro placental barrier construction using BeWo and HPVEC cells and articles' barrier crossing ability. FIG. 10B shows that the barrier formation was confirmed by measuring the TEER values and the article addition did not alter the TEER. FIG. 10C shows that the articles present in the apical side, inside the cells and the basolateral side were quantified using HPLC. The article concentrations were kept as 20 μg/mL with respect to ARTs and the incubation was conducted for 12 h. FIG. 10D shows the distribution of T-DRV-NP in the C57BL/6 pregnant mice, data presented in the % injected dose (% ID) and % injected dose per weight of the tissue (% ID/g).

FIG. 11 shows a synthetic scheme for Oc-[G-2]-(Pred)₄.

FIG. 12 shows cell viability of EFV, EVG, DRV, and their nanoformulations, tested at concentrations from 50 nM to 100 μM for 72 h in microglia

FIG. 13 shows elevated intracellular ROS levels in microglia cells treated with different concentrations of SQV, EFV, DRV, DTG, EVG or the nano-formulations T-SQV-NP, T-EFV-NP, T-DRV-NP, T-DTG-NP, T-EVG-NP for 24 h FIGS. 14A-14B show effects of (FIG. 14A) cocaine and (FIG. 14B) meth on mitochondrial basal respiration, maximal respiration, and ATP production as determined by Seahorse Mitostress analyses in astrocytes. The article concentration was varied from 0 to 500 μM and cells were treated for 24 h

FIGS. 15A-15C show distribution of intravenously injected T-ARV-NPs in the brain and blood of female Balb/c mice for (FIG. 15A) EFV and T-EFV-NP, (FIG. 15B) EVG and T-EVG-NP and (FIG. 15C) DRV and T-DRV-NP

FIGS. 16A-16B show Z_{average} hydrodynamic diameter, surface charge in terms of zeta potential, % loading and % encapsulation efficiency of T-CoQ₁₀-NP and T-(Asp)₄-NP.

FIG. 17 shows that HIV-infected human microglia cells were treated with drugs of abuse meth to elevate the responses further. The cells were then treated with T-EVG-NP or T-EFV-NP and then treated with T-(Asp)₄-NP and T-CoQ₁₀-NP. Inflammatory responses in cell supernatants by analyzing IL-1β.

FIG. 18 shows representative flow cytometry data and gating of Mito-GFP tagged microglia cells from non-stained cells FIG. 19 shows efficacy studies in EcoHIV infected Meth treated animal model. Levels of EcoHIV p24 antigen in the brain lysates by RT-PCR.

FIGS. 20A-20B shows expression of GFAP in isolated astrocytes and NeuN in isolated neurons by immunostaining method

FIG. 21 shows immunofluorescence images of brain tissues stained for p24 viral protein along with GFAP for astrocytes. C57BL/6 male mice were divided into 12 groups and were assigned to the following treatment groups: control virus+Meth+saline (12 animals); control virus+saline (12 animals); control virus+Meth+T-CoQ₁₀-NP/T-(Asp)₄-NP (13 animals); EcoHIV+Meth+saline (11 animals); EcoHIV+Meth+T-CoQ₁₀-NP/T-(Asp)₄-NP (10 animals); EcoHIV+Meth+T-ART-NPs (12 animals); EcoHIV+Meth+T-ART-NP+T-CoQ₁₀-NP/T-(Asp)₄-NP (16 animals); control Virus+Meth+T-CoQ₁₀/(Asp)₄-NPs (11 animals); control Virus+T-ART-NPs (13 animals); control Virus+T-ART-NPs+T-CoQ₁₀/(Asp)₄-NPs (12 animals), saline (3 animals), and EcoHIV+saline (3 animals). The mice were treated with either control virus (pBMN-I-GFP) or chimeric HIV-NDK (EcoHIV, 1 μg of p24), followed by one-week multiday exposure to Meth (0.2 mg/kg at each injection for 5 days) and subsequent two-week treatment with T-ART-NPs (5 mg/kg with respect to the drug) and T-CoQ₁₀/(Asp)₄-NPs (20 mg/kg with respect to the drug) via intravenous route. Scale bar=50 μm

FIG. 22 shows immunofluorescence images of brain tissues stained for p24 viral protein along with MAP2 for neurons. C57BL/6 male mice were divided into 12 groups and were assigned to the following treatment groups: control virus+Meth+saline (12 animals); control virus+saline (12 animals); control virus+Meth+T-CoQ₁₀-NP/T-(Asp)₄-NP (13 animals); EcoHIV+Meth+saline (11 animals); EcoHIV+Meth+T-CoQ₁₀-NP/T-(Asp)₄-NP (10 animals); EcoHIV+Meth+T-ART-NPs (12 animals); EcoHIV+Meth+T-ART-NP+T-CoQ₁₀-NP/T-(Asp)₄-NP (16 animals); control Virus+Meth+T-CoQ₁₀/(Asp)₄-NPs (11 animals); control Virus+T-ART-NPs (13 animals); control Virus+T-ART-NPs+T-CoQ₁₀/(Asp)₄-NPs (12 animals), saline (3 animals), and EcoHIV+saline (3 animals). The mice were treated with either control virus (pBMN-I-GFP) or chimeric HIV-NDK (EcoHIV, 1 μg of p24), followed by one-week multiday exposure to Meth (0.2 mg/kg at each injection for 5 days) and subsequent two-week treatment with T-ART-NPs (5 mg/kg with respect to the drug) and T-CoQ₁₀/(Asp)₄-NPs (20 mg/kg with respect to the drug) via intravenous route.

FIG. 23 shows immunofluorescence images of brain tissues stained for p24 viral protein along with TMEM119 for microglia. C57BL/6 male mice were divided into 12 groups and were assigned to the following treatment groups: control virus+Meth+saline (12 animals); control virus+saline (12 animals); control virus+Meth+T-CoQ₁₀-NP/T-(Asp)₄-NP (13 animals); EcoHIV+Meth+saline (11 animals); EcoHIV+Meth+T-CoQ₁₀-NP/T-(Asp)₄-NP (10 animals); EcoHIV+Meth+T-ART-NPs (12 animals); EcoHIV+Meth+T-ART-NP+T-CoQ₁₀-NP/T-(Asp)₄-NP (16 animals); control Virus+Meth+T-CoQ₁₀/(Asp)₄-NPs (11 animals); control Virus+T-ART-NPs (13 animals); control Virus+T-ART-NPs+T-CoQ₁₀/(Asp)₄-NPs (12 animals), saline (3 animals), and EcoHIV+saline (3 animals). The mice were treated with either control virus (pBMN-I-GFP) or chimeric HIV-NDK (EcoHIV, 1 μg of p24), followed by one-week multiday exposure to Meth (0.2 mg/kg at each injection for 5 days) and subsequent two-week treatment with T-ART-NPs (5 mg/kg with respect to the drug) and T-CoQ₁₀/(Asp)₄-NPs (20 mg/kg with respect to the drug) via intravenous route.

FIG. 24 shows immunofluorescence images of brain tissues stained for p24 viral protein along with ICAM-1 for neuroinflammation. C57BL/6 male mice were divided into 12 groups and were assigned to the following treatment groups: control virus+Meth+saline (12 animals); control virus+saline (12 animals); control virus+Meth+T-CoQ₁₀-NP/T-(Asp)₄-NP (13 animals); EcoHIV+Meth+saline (11 animals); EcoHIV+Meth+T-CoQ₁₀-NP/T-(Asp)₄-NP (10 animals); EcoHIV+Meth+T-ART-NPs (12 animals); EcoHIV+Meth+T-ART-NP+T-CoQ₁₀-NP/T-(Asp)₄-NP (16 animals); control Virus+Meth+T-CoQ₁₀/(Asp)₄-NPs (11 animals); control Virus+T-ART-NPs (13 animals); control Virus+T-ART-NPs+T-CoQ₁₀/(Asp)₄-NPs (12 animals), saline (3 animals), and EcoHIV+saline (3 animals). The mice were treated with either control virus (pBMN-I-GFP) or chimeric HIV-NDK (EcoHIV, 1 μg of p24), followed by one-week multiday exposure to Meth (0.2 mg/kg at each injection for 5 days) and subsequent two-week treatment with T-ART-NPs (5 mg/kg with respect to the drug) and T-CoQ₁₀/(Asp)₄-NPs (20 mg/kg with respect to the drug) via intravenous route.

FIG. 25 shows immunofluorescence images of brain tissues stained for p24 viral protein along with catalase for ROS marker. C57BL/6 male mice were divided into 12 groups and were assigned to the following treatment groups: control virus+Meth+saline (12 animals); control virus+saline (12 animals); control virus+Meth+T-CoQ₁₀-NP/T-(Asp)₄-NP (13 animals); EcoHIV+Meth+saline (11 animals); EcoHIV+Meth+T-CoQ₁₀-NP/T-(Asp)₄-NP (10 animals); EcoHIV+Meth+T-ART-NPs (12 animals); EcoHIV+Meth+T-ART-NP+T-CoQ₁₀-NP/T-(Asp)₄-NP (16 animals); control Virus+Meth+T-CoQ₁₀/(Asp)₄-NPs (11 animals); control Virus+T-ART-NPs (13 animals); control Virus+T-ART-NPs+T-CoQ₁₀/(Asp)₄-NPs (12 animals), saline (3 animals), and EcoHIV+saline (3 animals). The mice were treated with either control virus (pBMN-I-GFP) or chimeric HIV-NDK (EcoHIV, 1 μg of p24), followed by one-week multiday exposure to Meth (0.2 mg/kg at each injection for 5 days) and subsequent two-week treatment with T-ART-NPs (5 mg/kg with respect to the drug) and T-CoQ₁₀/(Asp)₄-NPs (20 mg/kg with respect to the drug) via intravenous route.

FIG. 26 shows H&E images of all the tissue sections after the treatment (Scale bar=50 μm). C57BL/6 male mice were divided into 12 groups and were assigned to the following treatment groups: control virus+Meth+saline (12 animals); control virus+saline (12 animals); control virus+Meth+T-CoQ₁₀-NP/T-(Asp)₄-NP (13 animals); EcoHIV+Meth+saline (11 animals); EcoHIV+Meth+T-CoQ₁₀-NP/T-(Asp)₄-NP (10 animals); EcoHIV+Meth+T-ART-NPs (12 animals); EcoHIV+Meth+T-ART-NP+T-CoQ₁₀-NP/T-(Asp)₄-NP (16 animals); control Virus+Meth+T-CoQ₁₀/(Asp)₄-NPs (11 animals); control Virus+T-ART-NPs (13 animals); control Virus+T-ART-NPs+T-CoQ₁₀/(Asp)₄-NPs (12 animals), saline (3 animals), and EcoHIV+saline (3 animals). The mice were treated with either control virus (pBMN-I-GFP) or chimeric HIV-NDK (EcoHIV, 1 μg of p24), followed by one-week multiday exposure to Meth (0.2 mg/kg at each injection for 5 days) and subsequent two-week treatment with T-ART-NPs (5 mg/kg with respect to the drug) and T-CoQ₁₀/(Asp)₄-NPs (20 mg/kg with respect to the drug) via intravenous route.

### DETAILED DESCRIPTION

In some aspects, disclosed herein are compositions and uses thereof for treating, preventing, inhibiting, and/or reducing HIV infection in a subject (e.g., a pregnant subject). In some embodiments, the compositions and methods disclosed herein can treat, prevent, inhibit, and/or reduce HIV infection in the central nervous system of the subject (e.g., a pregnant subject). Also disclosed herein are compositions and uses there for treating human immunodeficiency virus (HIV)-associated neurocognitive disorder (HAND) in a subject (e.g., a pregnant subject).

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of 20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The term “biocompatible” generally refers to a material and any metabolites or degradation products thereof that are generally non-toxic to the recipient and do not cause significant adverse effects to the subject.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “control” is an alternative subject or sample used in an experiment for comparison purposes. A control can be “positive” or “negative.”

The term “nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. For the purposes of the present invention, a nanoparticle typically ranges from about 1 nm to about 1000 nm, preferably from about 50 nm to about 500 nm, more preferably from about 50 nm to about 350 nm.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used herein, the term “antigen” refers to a molecule that is capable of binding to an antibody. In some embodiments, the antigen stimulates an immune response such as by production of antibodies specific for the antigen.

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” of expression or of activity are used to refer to inhibitory, activating, or modulating molecules, respectively, identified using in vitro and in vivo assays for expression or activity of a described target protein, e.g., antagonists and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Samples or assays comprising described target protein that are treated with a potential inhibitor are compared to control samples without the inhibitor to examine the extent of effect. Control samples (untreated with inhibitors) are assigned a relative activity value of 100%. Inhibition of a described target protein is achieved when the activity value relative to the control is about 80%, optionally 50% or 25, 10%, 5% or 1%.

The term “polypeptide” refers to a compound made up of a single chain of D- or L-amino acids or a mixture of D- and L-amino acids joined by peptide bonds.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, P A, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ).

The term “polymer” as used herein refers to a relatively high molecular weight organic compound, natural or synthetic, whose structure can be represented by a repeated small unit, the monomer. Synthetic polymers are typically formed by addition or condensation polymerization of monomers. The polymer is suitable for use in the body of a subject, i.e. is biologically inert and physiologically acceptable, non-toxic, and is biodegradable in the environment of use, i.e. can be resorbed by the body. The term “polymer” encompasses all forms of polymers including, but not limited to, natural polymers, synthetic polymers, homopolymers, heteropolymers or copolymers, addition polymers, etc.

The term “copolymer” as used herein refers to a polymer formed from two or more different repeating units (monomer residues). Copolymer compasses all forms copolymers including, but not limited to block polymers, random copolymers, alternating copolymers, or graft copolymers. A “block copolymer” is a polymer formed from multiple sequences or blocks of the same monomer alternating in series with different monomer blocks. Block copolymers are classified according to the number of blocks they contain and how the blocks are arranged.

The term “subject” is defined herein to include animals such as mammals, including, but not limited to, primates (e.g., humans), cows, sheep, goats, horses, dogs, cats, rabbits, rats, mice and the like. In some embodiments, the subject is a human. In some embodiments, the subject is a pregnant subject.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g., a composition comprising an agent) refers to an amount that is effective to achieve a desired therapeutic result. In some embodiments, a desired therapeutic result is the control of viral levels. In some embodiments, a desired therapeutic result is the control of HAND, or a symptom of HAND. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The terms “treat,” “treating,” “treatment,” and grammatical variations thereof as used herein, include partially or completely delaying, alleviating, mitigating or reducing the intensity of one or more attendant symptoms of HIV infection or condition and/or alleviating, mitigating or impeding one or more symptoms of HIV infection. Treatments according to the invention may be applied preventively, prophylactically, palliatively or remedially. Prophylactic treatments are administered to a subject prior to onset (e.g., before obvious signs of an infection), during early onset (e.g., upon initial signs and symptoms of an infection), after an established development of an infection, or during chronic infection. Prophylactic administration can occur for several minutes to months prior to the manifestation of an infection.

### Compositions

In some aspects, disclosed herein is a nanoparticle comprising:
a mitochondrial targeting moiety;
an anti-HIV therapeutic agent; and/or
one or more of an anti-oxidant agent and/or an anti-inflammatory agent.

In some aspects, disclosed herein is a nanoparticle comprising:
a mitochondrial targeting moiety;
an anti-HIV therapeutic agent; and
one or more of an anti-oxidant agent and/or an anti-inflammatory agent.

In some aspects, disclosed herein is a nanoparticle comprising:
a mitochondrial targeting moiety; and
an anti-HIV therapeutic agent.

In some aspects, disclosed herein is a nanoparticle comprising:
a mitochondrial targeting moiety; and
one or more of an anti-oxidant agent and/or an anti-inflammatory agent.

In some embodiments, the anti-HIV therapeutic agent and the one or more of an anti-oxidant agent and/or an anti-inflammatory agent can be found within one nanoparticle. In some embodiments, the anti-HIV therapeutic agent and the one or more of an anti-oxidant agent and/or an anti-inflammatory agent can be found within separate nanoparticles (for example, the composition comprises nanoparticles loaded with an anti-HIV therapeutic agent, and separate nanoparticles loaded with an anti-oxidant agent and/or nanoparticles loaded with an anti-inflammatory agent).

In some aspects, disclosed herein is a composition comprising:
a nanoparticle comprising a mitochondrial targeting moiety;
a nanoparticle comprising an anti-HIV therapeutic agent; and/or
a nanoparticle comprising one or more of an anti-oxidant agent and/or an anti-inflammatory agent.

In some aspects, disclosed herein is a composition comprising:
a nanoparticle comprising a mitochondrial targeting moiety;
a nanoparticle comprising an anti-HIV therapeutic agent; and
a nanoparticle comprising one or more of an anti-oxidant agent and/or an anti-inflammatory agent.

In some aspects, disclosed herein is a composition comprising:
a nanoparticle comprising a mitochondrial targeting moiety; and
a nanoparticle comprising an anti-HIV therapeutic agent.

In some aspects, disclosed herein is a composition comprising:
a nanoparticle comprising a mitochondrial targeting moiety; and
a nanoparticle comprising one or more of an anti-oxidant agent and/or an anti-inflammatory agent.

In some embodiments, the nanoparticles comprising the different components above are administered in one composition. In some embodiments, the nanoparticles comprising the different components above are administered in separate compositions. In some embodiments, the nanoparticles comprising the different components are administered simultaneously. In some embodiments, the nanoparticles comprising the different components are administered at different time points.

The nanoparticles described herein include one or more moieties that target the nanoparticles to mitochondria. As used herein, “targeting” a nanoparticle to mitochondria means that the nanoparticle accumulates in mitochondria relative to other organelles or cytoplasm at a greater concentration than substantially similar non-targeted nanoparticle. A substantially similar non-target nanoparticle includes the same components in substantially the same relative concentration as the targeted nanoparticle, but lacks a targeting moiety.

Any suitable moiety for facilitating accumulation of the nanoparticle within the mitochondrial matrix may be employed. Nanoparticles having a mitochondrial targeting moiety may be made in any suitable manner. In some embodiments, nanoparticles can be constructed as described in (i) WO 2013/123298, published on Aug. 22, 2012, entitled Nanoparticles for Mitochondrial Trafficking of Agents, and describing information generally as disclosed in Marrache and Dhar (Oct. 2, 2012), Proc. Natl. Acad. Sci. USA, vol. 109 (40), pages 16288-16293; or (ii) WO 2013/033513, published on Mar. 7, 2013, entitled Apoptosis-Targeting Nanoparticles, which claims priority to U.S. Provisional Patent Application No. 61/529,637 filed on Sep. 9, 2012, each of which patent applications and publications are incorporated herein by reference in their respective entireties to the extent that they do not conflict with the present disclosure. Triphenyl phosphonium (TPP) containing compounds can accumulate greater than 1000 fold within the mitochondrial matrix. In some embodiments, the mitochondrial targeting moiety comprises a terminal triphenylphosphonium (TPP) cation. In some embodiments, the mitochondrial targeting moiety comprises a triphenyl phosphonium (TPP) moiety or a derivative thereof. TPP cation or triphenyl phosphonium (TPP) moiety is known in the art. See, e.g., U.S, Patent Publication NO: US20170216219A1, incorporated by reference herein in its entirety.

In some embodiments, the nanoparticle has a diameter from about 1 nm to about 1000 nm. In some embodiments, the nanoparticle has a diameter less than, for example, about 1000 nm, about 950 nm, about 900 nm, about 850 nm, about 800 nm, about 750 nm, about 700 nm, about 650 nm, about 600 nm, about 550 nm, about 500 nm, about 450 nm, about 400 nm, about 350 nm, about 300 nm, about 290 nm, about 280 nm, about 270 nm, about 260 nm, about 250 nm, about 240 nm, about 230 nm, about 220 nm, about 210 nm, about 200 nm, about 190 nm, about 180 nm, about 170 nm, about 160 nm, about 150 nm, about 140 nm, about 130 nm, about 120 nm, about 110 nm, about 100 nm, about 90 nm, about 80 nm, about 70 nm, about 60 nm, about 50 nm, about 40 nm, about 30 nm, about 20 nm, or about 10 nm. In some embodiments, the nanoparticle has a diameter, for example, from about 20 nm to about 1000 nm, from about 20 nm to about 800 nm, from about 20 nm to about 700 nm, from about 30 nm to about 600 nm, from about 30 nm to about 500 nm, from about 40 nm to about 400 nm, from about 40 nm to about 300 nm, from about 40 nm to about 250 nm, from about 50 nm to about 250 nm, from about 50 nm to about 200 nm, from about 50 nm to about 150 nm, from about 60 nm to about 150 nm, from about 70 nm to about 150 nm, from about 80 nm to about 150 nm, from about 90 nm to about 150 nm, from about 100 nm to about 150 nm, from about 110 nm to about 150 nm, from about 120 nm to about 150 nm, from about 90 nm to about 140 nm, from about 90 nm to about 130 nm, from about 90 nm to about 120 nm, from 100 nm to about 140 nm, from about 100 nm to about 130 nm, from about 100 nm to about 120 nm, from about 100 nm to about 110 nm, from about 110 nm to about 120 nm, from about 110 nm to about 130 nm, from about 110 nm to about 140 nm, from about 90 nm to about 200 nm, from about 100 nm to about 195 nm, from about 110 nm to about 190 nm, from about 120 nm to about 185 nm, from about 130 nm to about 180 nm, from about 140 nm to about 175 nm, from 150 nm to 175 nm, or from about 150 nm to about 170 nm. In some embodiments, the nanoparticle has a diameter from about 100 nm to about 250 nm. In some embodiments, the nanoparticle has a diameter from about 150 nm to about 175 nm. In some embodiments, the nanoparticle has a diameter from about 135 nm to about 175 nm. The particles can have any shape but are generally spherical in shape.

Any suitable synthetic or natural biocompatible polymers may be used. Such polymers are recognizable and identifiable by one or ordinary skill in the art. The polymers used for nanoparticles are known in the art. See, e.g., U.S, Patent Publication NO: US20170216219A1, incorporated by reference herein in its entirety. In some embodiments, the nanoparticle comprise poly(lactic-co-glycolic acid) (PLGA)-block(b)-polyethylene glycol (PEG). In some embodiments the nanoparticle comprises a PLGA-PEG-TPP based polymer.

The amount of a therapeutic agent that can be present in the nanoparticle can be from about 0.1% to about 90% of its nanoparticle weight. For example, the amount of a therapeutic agent present in the nanoparticle can be from about 0.1%, about 0.2%, about 0.3%, about 0.4%, about 0.5%, about 0.6%, about 0.7%, about 0.8%, about 0.9%, about 1%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 15%, about 16%, about 17%, about 18%, about 19%, about 20%, about 22%, about 24%, about 26%, about 28%, about 30%, about 32%, about 34%, about 36%, about 38%, about 40%, about 42%, about 44%, about 46%, about 50%, about 55%, about 60%, about 65%, about 70%, about 75%, or about 80% of its nanoparticle weight.

In some embodiments, the nanoparticle comprises the anti-oxidant agent and the anti-inflammatory agent. In some embodiments, the nanoparticle comprises the anti-oxidant agent. In some embodiments, the nanoparticle comprises the anti-inflammatory agent. In some embodiments, the nanoparticle comprises the anti-HIV therapeutic agent. Any suitable antioxidant may be used. Examples of antioxidants include glutathione, vitamin C, vitamin A, vitamin E, calalase, superoxise dismutate, a peroxidase, coenzyme Q10 (CoQ₁₀), and the like. In some embodiments, the anti-oxidant comprises Coenzyme Q10 (CoQ₁₀).

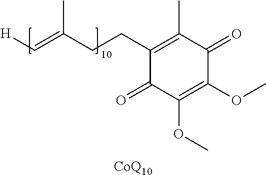

Any suitable anti-inflammatory agent may be used. In some embodiments, the anti-inflammatory agent comprises a prodrug of aspirin or prednisone. In some embodiments, the prodrug of aspirin is Oc-G2-(Asp)₄ (Asp4). In some embodiments, the prodrug of prednisone is Oc-[G2]-(Pred)₄.

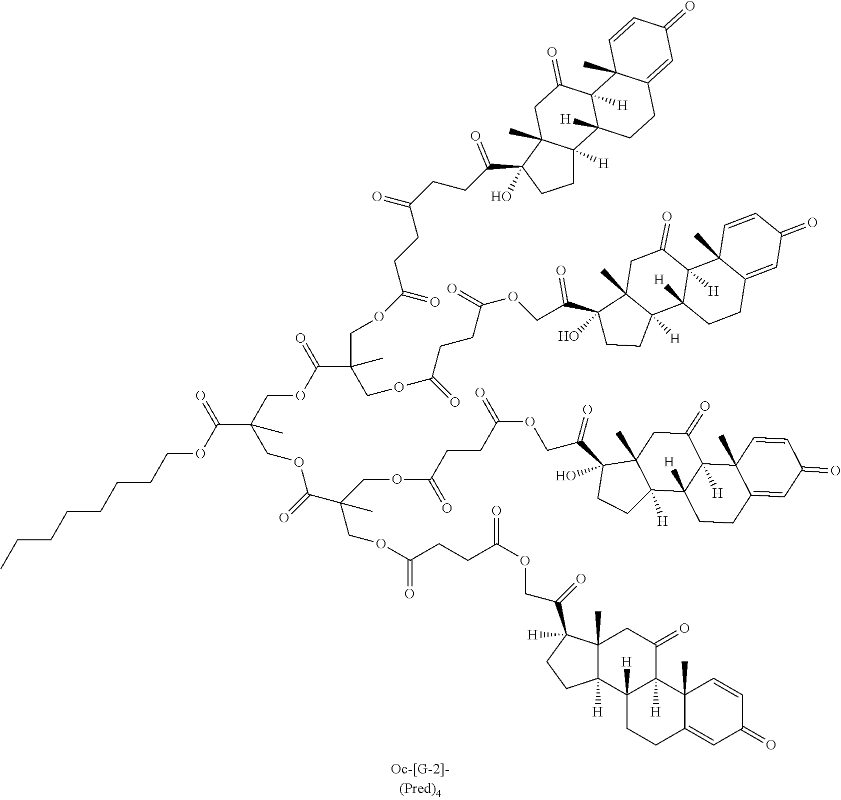

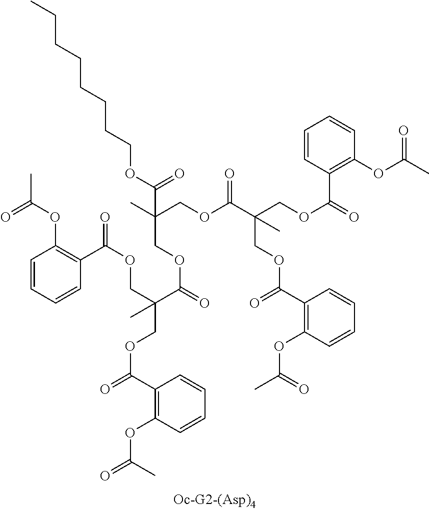 [SMILES: CCCCCCCCOC(=O)C(C)(COC(=O)C(C)(COC(=O)c1ccccc1OC(C)=O)COC(=O)c1ccccc1OC(C)=O)COC(=O)C(C)(COC(=O)c1ccccc1OC(C)=O)COC(=O)c1ccccc1OC(C)=O]

In some embodiments, the anti-HIV therapeutic agent comprises a protease inhibitor, an integrase inhibitor, a fusion inhibitor, a nucleoside reverse transcriptase inhibitor (NRTI), a nucleotide reverse transcriptase inhibitor (NtRTI), a non-nucleoside reverse-transcriptase inhibitor (NNRTI), a CCR5 antagonist, a post attachment inhibitor, or a maturation inhibitor.

In some embodiments, the integrase inhibitor comprises raltegravir, elvitegravir, dolutegravir, bictegravir, V-165, BI 224436, MK-2048, Lens epithelial derived growth factor LEDGF, or cabotegravir.

In some embodiments, the protease inhibitor comprises amprenavir, lopinavir Kaletra, saquinavir, indinavir, ritonavir, nelfinavir, atazanavir, fosamprenavir, tipranavir, darunavir, DMP450, PNU-140690, ABT-378, PD178390, asunaprevir, boceprevir, grazoprevir, glecaprevir, paritaprevir, simeprevir, telaprevir, tanomastat, batimastat, or bortezomib.

In some embodiments, the NRTI comprises zidovudine, didanosine, didanosine EC, zalcitabine, stavudine, lamivudine, abacavir, emtricitabine, entecavir, or Truvada.

In some embodiments, the NtRTI comprises tenofovir or adefovir.

In some embodiments, the NNRTI comprises efavirenz, nevirapine, delavirdine, etravirine, rilpivirine, or doravirine.

In some embodiments, the CCR5 antagonist comprises leronlimab, aplaviroc, vicriviroc, maraviroc, or INCB009471.

In some embodiments, the fusion inhibitor comprises efuvirtide.

In some embodiments, the anti-HIV therapeutic agent comprises saquinavir, darunavir, elvitegravir, dolutegravir, raltegravir, bictegravir, efavirenz, delavirdine, or stavudine.

In some embodiments, the anti-HIV therapeutic agent comprises saquinavir, efavirenz, or elvitegravir.

In some embodiments, the nanoparticle of any preceding aspect crosses a blood brain barrier. In some embodiments, the nanoparticle does not cross a placental barrier.

In some aspects, disclosed herein is a pharmaceutical composition comprising one or more of the nanoparticles described herein. In some embodiments, the nanoparticle comprises an anti-HIV therapeutic agent. In some embodiments, the nanoparticle comprises an anti-oxidant agent. In some embodiments, the nanoparticle comprises an anti-inflammatory agent.

It should be understood that the compositions disclosed herein can cross blood-brain-barrier and accumulate in central nervous systems. Brain accumulation happens due to several parameters: 1) because the particles are smaller in size; 2) because the particles have a lipophilic delocalized positive surface; 3) because the endothelial cells at the blood brain barrier have mitochondria than other types of endothelial cells in the body and hence the mitochondria targeting ability contributes positively to get the particles to the brain. Further, the endothelial cells at the blood brain barrier are more sulfated rather than being phosphate and hence the positive lipophilic surface helps; 4) once in the brain, the particles can stay by interacting with glia cells because these cells have hyperpolarized mitochondria, hence mitochondria targeting properties of the particles help; 5) the brush structure of the nanoparticle helps and the brush structure is created because of the way these particles are made by creating a dense surface.

### Methods

Disclosed herein is a method for reducing a level of human immunodeficiency virus (HIV) in the central nervous system of a subject (e.g., a pregnant subject) in need thereof, comprising administering to the subject (e.g., a pregnant subject) a therapeutically effective amount of the compound or a therapeutically effective amount of the nanoparticle disclosed herein.

Disclosed herein is a method for treating human immunodeficiency virus (HIV)-associated neurocognitive disorder (HAND) in a subject (e.g., a pregnant subject) in need thereof, comprising administering to the subject (e.g., a pregnant subject) a therapeutically effective amount of the nanoparticle disclosed herein.

The present disclosure shows that the nanoparticle composition described herein can cross blood brain barrier but does not cross blood placental barrier. In some embodiments, the subject is a human. In some embodiments, the subject is pregnant. In some embodiments, the subject uses a recreational drug.

Also disclose herein is a method for reducing a level of neuroinflammation caused by human immunodeficiency virus (HIV) in the central nervous system of a subject in need thereof, comprising administering a therapeutically effective amount of the nanoparticle disclosed herein.

Also disclosed herein is a method for reducing a level of neuroinflammation in the brain or central nervous system of a subject in need thereof, comprising administering a therapeutically effective amount of the nanoparticle disclosed herein.

Also disclosed herein is a method for reducing a level of oxidative stress caused by human immunodeficiency virus (HIV) in the central nervous system of a subject in need thereof, comprising administering a therapeutically effective amount of the nanoparticle disclosed herein.

Also disclosed herein is a method for reducing a level of oxidative stress in the brain or central nervous system of a subject in need thereof, comprising administering a therapeutically effective amount of the nanoparticle disclosed herein.

Also disclosed herein is method for reducing a level of oxidative stress caused by antiretroviral drugs used for treatment of human immunodeficiency virus (HIV) in the central nervous system of a subject in need thereof, comprising administering a therapeutically effective amount of the nanoparticle disclosed herein.

Also disclosed herein is a method for reducing a level of inflammation or neuroinflammation caused by antiretroviral drugs used for treatment of human immunodeficiency virus (HIV) in the central nervous system of a subject in need thereof, comprising administering a therapeutically effective amount of the nanoparticle disclosed herein.

Also disclosed herein is a method for protecting neurons by reducing a level of inflammation or neuroinflammation in the central nervous system of a subject in need thereof, comprising administering a therapeutically effective amount of the nanoparticle disclosed herein.

Also disclosed herein is a method for protecting neurons by reducing a level of inflammation or neuroinflammation in the central nervous system of a subject in need thereof, comprising administering a therapeutically effective amount of the nanoparticle disclosed herein.

Also disclosed herein is a method for protecting neurons by reducing a level of oxidative stress in the central nervous system of a subject in need thereof, comprising administering a therapeutically effective amount of the nanoparticle disclosed herein.

Also disclosed herein is a method for protecting microglia by reducing a level of oxidative stress and/or inflammation in the central nervous system of a subject in need thereof, comprising administering a therapeutically effective amount of the nanoparticle disclosed herein.

Also disclosed herein is a method for protecting astrocytes by reducing a level of oxidative stress and/or inflammation in the central nervous system of a subject in need thereof, comprising administering a therapeutically effective amount of the nanoparticle disclosed herein.

Also disclosed herein is a method for reducing a level of oxidative stress and inflammation caused by human immunodeficiency virus (HIV) and/or abuse drugs such as methamphetamine, cocaine, and/or opioids in the central nervous system of a subject in need thereof, comprising administering a therapeutically effective amount of the nanoparticle disclosed herein.

Also disclosed herein is a method for reducing a level of oxidative stress and inflammation caused by abuse drugs such as methamphetamine, cocaine, or opioids or morphine in the central nervous system of a subject in need thereof, comprising administering a therapeutically effective amount of the nanoparticle disclosed herein.

Also disclosed herein is a method for reducing a level of oxidative stress and inflammation caused by human immunodeficiency virus (HIV) and/or abuse drugs such as methamphetamine or cocaine or opioids in the central nervous system of a pregnant women in need thereof, comprising administering a therapeutically effective amount of the nanoparticle disclosed herein.

The amounts of an anti-HIV therapeutic agent dispersed or encapsulated in the nanoparticle or adhering to the nanoparticle composition described herein can be generally smaller, e.g., at least about 10% smaller, than the amount of the anti-HIV therapeutic agent present in the current dosage of the treatment regimen (i.e., without the nanoparticle composition) required for producing essentially the same therapeutic effect. Indeed, an anti-HIV therapeutic agent encapsulated in, or adhered to, the nanoparticle composition can potentially increase duration of the therapeutic effect for the anti-HIV therapeutic agent. Stated another way, encapsulating an anti-HIV therapeutic agent in the nanoparticle or adhering an anti-HIV therapeutic agent to the nanoparticle can increase its therapeutic efficacy, i.e., a smaller amount of the anti-HIV therapeutic agent encapsulated in the nanoparticle, as compared to the amount present in a typical one dosage administered for an HIV or HAND patient, can achieve essentially the same therapeutic effect. Accordingly, the nanoparticle composition can comprise an anti-HIV therapeutic agent in an amount which is less than the amount traditionally recommended for one dosage of the anti-HIV therapeutic agent, while achieving essentially the same therapeutic effect. For example, if the traditionally recommended dosage of an anti-HIV therapeutic agent is X amount then the nanoparticle composition described herein can comprise the anti-HIV therapeutic agent in an amount of about 0.9×, about 0.8×, about 0.7×, about 0.6×, about 0.5×, about 0.4×, about 0.3×, about 0.2×, about 0.1× or less. Without wishing to be bound by the theory, this can allow administering a lower dosage of the anti-HIV therapeutic agent in the nanoparticle to obtain a therapeutic effect which is similar to when a higher dosage is administered without the nanoparticle composition. Low-dosage administration of an anti-HIV therapeutic agent can reduce side effects of the anti-HIV therapeutic agent, if any, and/or reduce likelihood of the subject's resistance to the anti-HIV therapeutic agent after administration for a period of time.

Dosing frequency for the nanoparticle or the nanoparticle drug composition disclosed herein, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiments, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiments, the dosing frequency for the nanoparticle composition includes, but is not limited to, at least once a day, twice a day, or three times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiments, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiments, the interval between each administration is constant. For example, the administration can be carried out daily, every two days, every three days, every four days, every five days, or weekly. Administration can also be continuous and adjusted to maintaining a level of the compound within any desired and specified range.

The disclosed methods can be performed any time prior to or during pregnancy. In some aspects, the disclosed compositions or methods can be employed 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, or2 weeks prior to pregnancy. In some embodiments, the composition disclosed herein is administered beginning about the 18^{th} to 22^{nd} week of gestation until about the 37^{th} week of gestation, or for approximately 14 to 19 weeks, depending on the gestational age at the beginning of treatment and the date of delivery. In some embodiments, the composition disclosed herein is administered beginning about the 16^{th} week of gestation until about the 37^{th} week of gestation, or for approximately 21 weeks. In some embodiments, the composition disclosed herein is administered beginning about the time of a positive pregnancy test until about the 37^{th} week of gestation or beginning about the 2nd to 4th week of gestation, for approximately 33 to 35 weeks. In some embodiments, the composition disclosed herein is administered beginning about the time of a positive pregnancy test until about the 39th week of gestation. In some embodiments, the composition disclosed herein is administered prior to pregnancy and through pregnancy.

Accordingly, also disclosed herein is a method for reducing a level of human immunodeficiency virus (HIV) in a subject trying to get pregnant in need thereof, comprising administering to the pregnant subject a therapeutically effective amount of the nanoparticle, wherein the nanoparticle comprises:

a mitochondrial targeting moiety;

an anti-HIV therapeutic agent; and/or

one or more of an anti-oxidant agent and/or an anti-inflammatory agent.

In some embodiments, the administration of the nanoparticle reduces a level of HIV in the central nervous system and/or blood of the pregnant subject or subject who is planning to become pregnant.

### EXAMPLES

The following examples are set forth below to illustrate the compositions, nanoparticles, methods, and results according to the disclosed subject matter. These examples are not intended to be inclusive of all aspects of the subject matter disclosed herein, but rather to illustrate representative systems, methods, compositions and results. These examples are not intended to exclude equivalents and variations of the present invention which are apparent to one skilled in the art.

### Example 1. Brain Accumulating Nanoparticles for Assisting Astrocytes to Reduce Human Immunodeficiency Virus and Drug Abuse-Induced Neuroinflammation and Oxidative Stress

The use of combination antiretroviral therapy (cART) has changed the acute epidemic of the human immunodeficiency virus (HIV) infections and acquired immunodeficiency syndrome (AIDS) into a chronic and treatable disease. More than ˜690 K people lose their lives from AIDS each year and in 2019, 1.7 million people became newly infected with HIV. The hard-to-reach HIV reservoirs in the brain are often untreatable due to the inefficiency of ART in crossing the blood-brain barrier (BBB) to reach HIV-infected cells in the brain. Failure to treat these reservoirs often leads to severe neurological problems, named HIV-associated neurocognitive disorders (HAND), such as HIV dementia. In addition, a large percentage of HIV-positive individuals abuse drugs such as methamphetamine (meth) and cocaine, which cause further oxidative stress by enhancing reactive oxygen species (ROS), mitochondrial dysfunctions, and inflammatory processes in HIV-infected areas in the brain. Therefore, there is a growing need for ART not only to be delivered across the BBB, but to be paired with antioxidant and/or anti-inflammatory neuroprotectants to alleviate HAND caused by both ART and drugs of abuse in the brains of HIV-positive individuals. Although the precise underlying neuropathological mechanisms of HIV in humans are not completely understood, alterations in the function of glial cells such as astrocytes have been correlated with impaired neuronal function, including detrimental changes in synaptic function, neuronal polarity, axon and dendrite formation, and neuronal survival. Furthermore, the activation of microglia and macrophages for neuro-inflammation, the onset of mitochondrial dysfunction, and the formation of ROS in astrocytes impair the neuroprotective abilities of astrocytes. Most of the therapeutic options, such as ART, that are meant to treat the brain viral reservoirs as well as the neuroprotectants intended to tackle HAND, have limited efficacy due to the inability of the majority of these agents to cross the BBB, even when the barrier is partially compromised by infection. A refined quality of life and overall survival for patients living with HIV infection urgently requires innovative modalities of combination treatments.

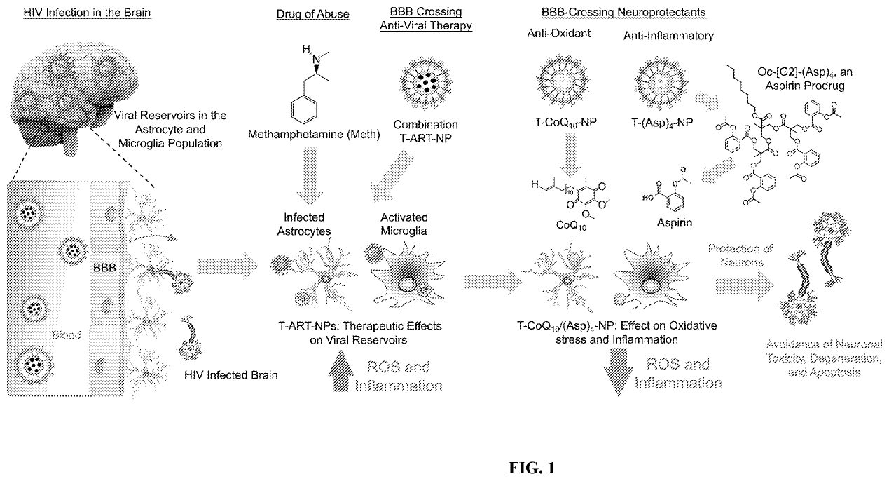

Nanoparticle (NP)-mediated delivery of cART and supplementation with antioxidant/anti-inflammatory-based neuroprotectants to the brain to improve neuronal functions for HIV-drug abuse conditions has not achieved its full potential. The delivery of antiretroviral (ARV) drugs and neuroprotectants to the brain can potentially be achieved by using bio-degradable NPs engineered to achieve the following crucial milestones: (1) optimized size, charge, lipophilicity, and targeting properties to cross the BBB; (2) ability to reach the viral reservoirs and specific intracellular targets while demonstrating controlled release of the payload at the target and without toxicity; and (3) ameliorative effects on infected are-as. This would enable effective and robust delivery of ARVs to the viral reservoirs and neuroprotectants to target cells such as astrocytes and microglia, to prevent neuronal degeneration. Thus, a highly lipophilic, biodegradable NP delivery platform with the ability to cross the BBB and deliver (1) ARVs to viral reservoirs of the brain, (2) anti-oxidants to the mitochondrial matrix of astrocytes rich in ROS, and (3) anti-inflammatory agents to the microglia of the brain can be extremely beneficial and is urgently needed to show therapeutic effects and to control HAND in HIV-infected patients. In this work, it is shown that a combination of a brain-accumulating, mitochondrion-targeted, polymeric nanoparticle (T-NP) separately containing optimized combinatory ARVs, a mitochondrion-acting anti-oxidant, coenzyme Q10 (CoQ10), and a prodrug [Oc-G2-(Asp)₄] of an FDA-approved anti-inflammatory agent, aspirin, has the potential to provide therapeutic intervention against the viral load in the brain and tackle inflammation, oxidative stress, and mitochondrial dysfunction simultaneously in presence of the virus and the recreational drug meth (FIG. 1).

### Results.

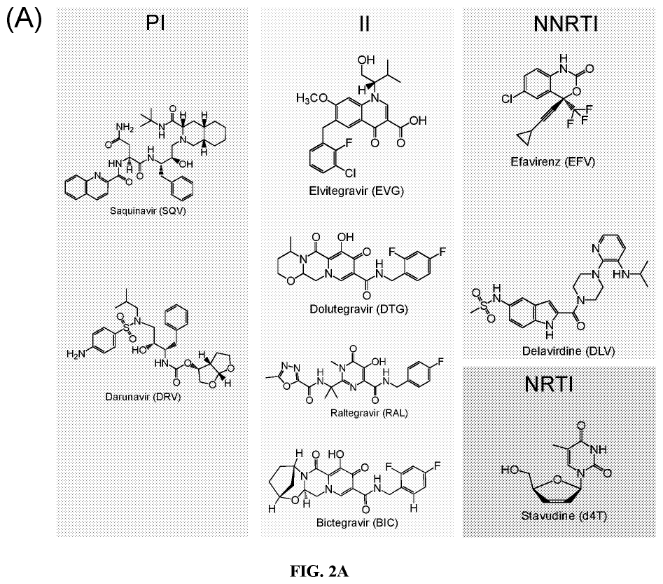

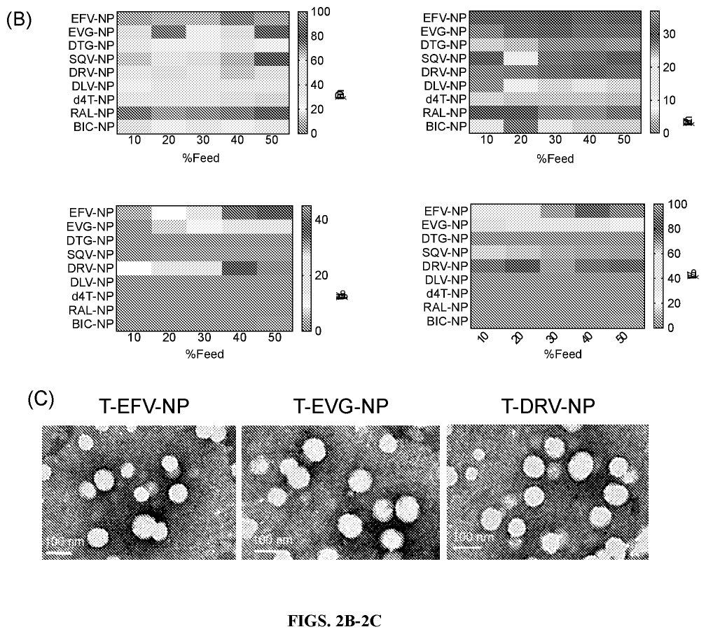

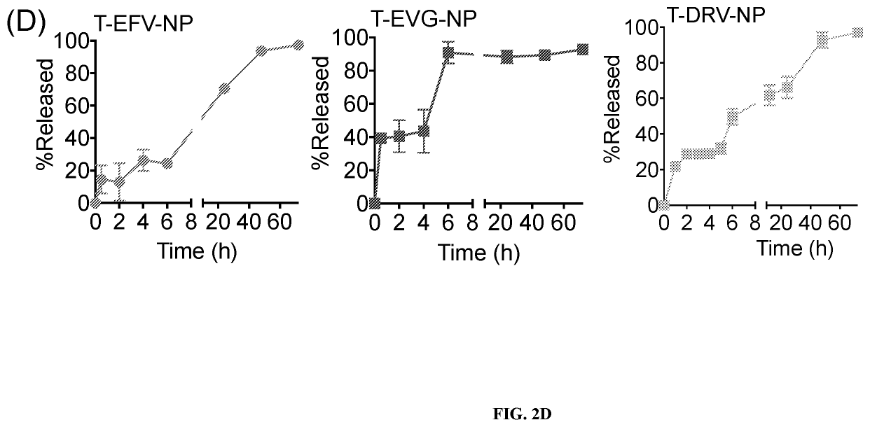

Optimization of T-ARV-NPs. It was recently demonstrated that a self-assembled NP originating from a biocompatible block copolymer, poly(D,L-lactic-co-glycolic acid)-block-poly(ethyleneglycol)-triphenylphosphonium (PLGA-b-PEG-TPP) when blended with 10% TPP-(CH₂)₅—COOH has the ability to cross the BBB based on in vivo studies conducted in small and large animal as well as in vitro BBB models. A library of ARV loaded brain accumulating NPs were first set out for optimization. The addition of 10% TPP-(CH₂)₅—COOH did not result in immunogenicity or toxicity from the NPs but did significantly improve uptake into the brain. The initial loading of 26 most widely used ARV drugs was tested in NPs of the targeted PLGA-b-PEG-TPP polymer at 20% feed of the drug with respect to the polymer (Table 1). The NPs were prepared using the conventional nanoprecipitation technique and purified by ultracentrifugation method with a filter of 100 kDa molecular weight cutoff. The Z-average hydrodynamic diameter and surface charge in terms of zeta potential of the NPs were measured using dynamic light scattering (DLS) technique. The compiled data represented in Table 1 indicated that the diameter of the ART encapsulated NPs is in the range of 50-70 nm and the surface is positively charged. To test for the amount of drug-loaded into the NPs, the NPs were subjected to high performance liquid chromatography (HPLC) along with free drug standards. The area under the curve was compared between standards and the NPs to find out the concentration of an encapsulated ARV. Of the 26 drugs, only 9 drugs, saquinavir (SQV), darunavir (DRV), elvitegravir (EVG), dolutegravir (DTG), raltegravir (RAL), bictegravir (BIC), efavirenz (EFV), delavirdine (DLV), and stavudine (d4T) showed signs of loading into the NPs (Table 1). Five of these drugs, DTG, RAL, DLV, BIC, and d4T showed very minimal loading. The 4 drugs that showed a higher percentage of loading were SQV, EFV, DRV, and EVG (Table 1). Further optimization of NPs on multiple fronts was then carried out. First, the % feed of ARVs was varied to look for the best possible loading and encapsulation efficiency (EE) for a given drug in the NPs keeping the NP diameter below 100 nm and a zeta potential indicating a highly positive charged surface. Next, the stability of NPs with respect to drug release profiles was investigated. Typically, with these types of formulations, the drug release profiles from hours to days can be tuned. Selected formulations were evaluated for drug release kinetics under physiological conditions of pH 7.4 at 37° C. These studies examined the % loading of 9 different ARTs from four different categories: protease inhibitors (PI), which inhibit HIV's protease and final proteolytic cleavage of viral protein precursors; integrase inhibitors (II), which prevent insertion of HIV viral genome into host cell DNA; and nucleoside and non-nucleoside reverse transcriptase inhibitor (NRTI, NNRTI), which block HIV's reverse transcriptase enzyme from con-verting viral RNA to DNA (FIG. 2A). This was done by varying the percent feed from 10 to 50% and indicated that three drugs, EVF, EVG, and DRV, showed improved loading and NPs with good physicochemical properties (FIG. 2B). Thus, these three drugs were selected to be incorporated in the T-NPs for application under conditions of HIV and drugs of abuse. Since the optimization techniques resulted in good NPs encapsulation of drug from each category, this work investigates the effects of a combination of DRV, which is a PI, EVG, which is an II, and EFV, which is a NNRTI (FIG. 2A). Analysis of the morphology of the NPs encapsulated with these three drugs by transmission electron microscopy (TEM) indicated spherical, homogeneous particles (FIG. 2C) complementing the diameter determined by DLS technique. In vitro drug release studies in PBS (pH-7.4) at 37° C. indicated controlled release profiles of all the three drugs from the respective NPs (FIG. 2D).

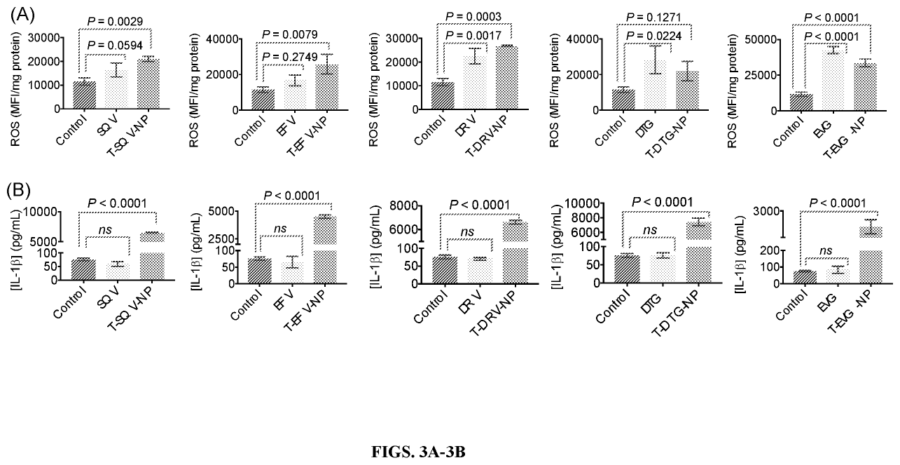

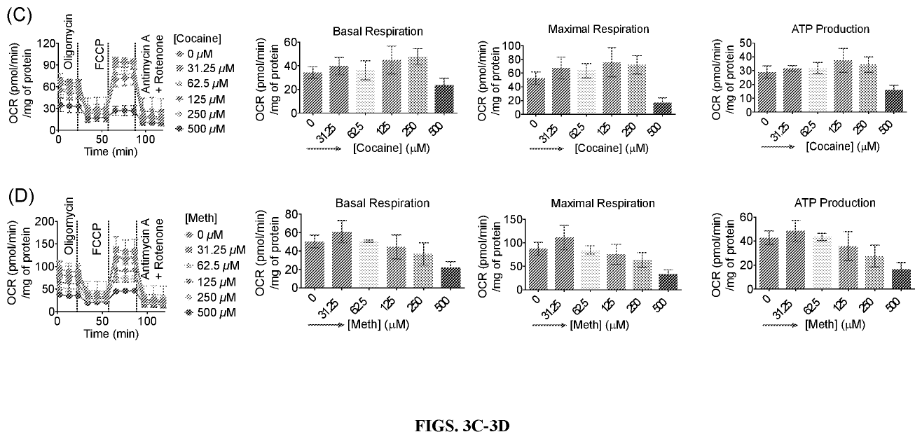

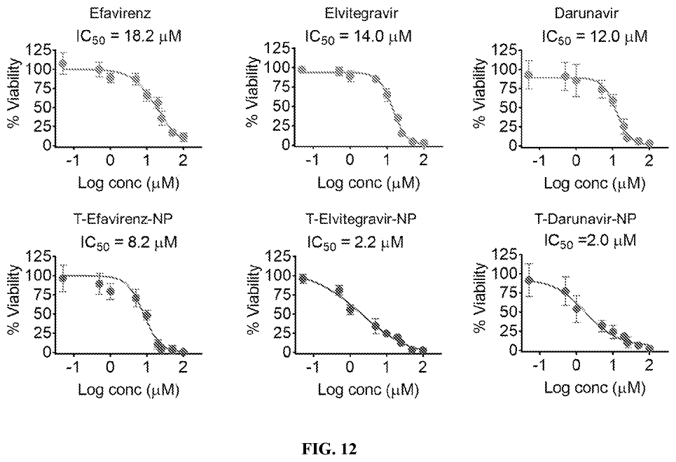

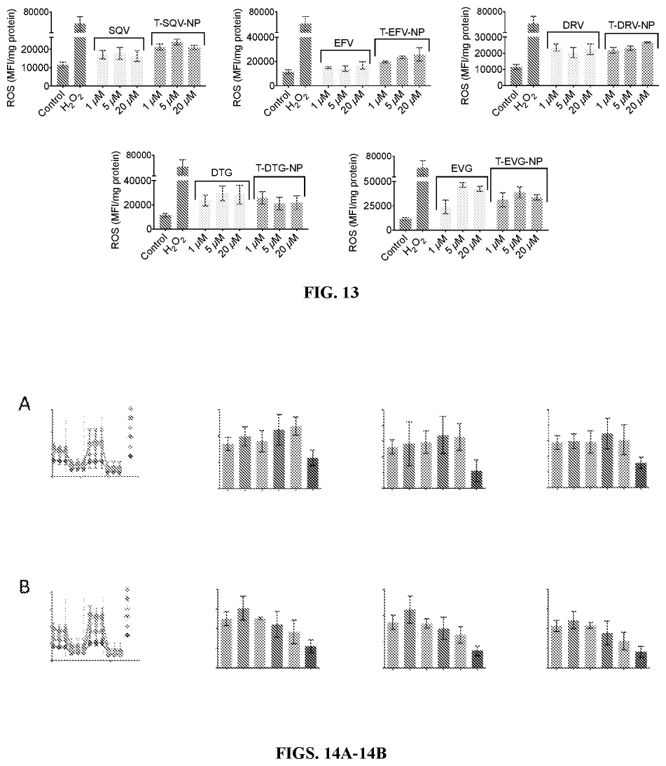

Effects ofARTs and Drugs ofAbuse on Cellular Health. HIV-associated neurocognitive disorders affecting glia cells become worse when the HIV patients use drugs of abuse such as cocaine and meth. In addition, well-known ARVs also contribute to inflammation and oxidative stress to worsen the symptoms of HAND. For example, EFV has been shown to have effects in the CNS such as dizziness, impaired concentration, and sleep disturbance. EFV can have damaging effects on neurons and microglia by altering calcium homeostasis, decreasing brain creatine kinase, and increasing pro-inflammatory cytokine levels, through mitochondrial damage. Changes in bioenergetics are caused by inhibition of mitochondrial complex IV of the electron transport chain (ETC). EVG is also associated clinical psychiatric symptoms. EVG has been shown to have similar toxic effects on neurons and microglia, along with other side effects such as diarrhea. These toxic effects increase when a NP-based delivery approach is utilized since with nanoparticle delivery of ARVs, these are delivered specifically to the brain. Thus, by using aspirin- and CoQ10-containing NPs along with ARV-NPs, these effects can be mitigated while keeping the drugs' antiretroviral activity intact. The toxic effects of the ARTs, which were loaded in the NPs at an appreciable amount, were checked. Microglia cells were treated with EFV, EVG, and DRV or the respective ARV-loaded NPs at various concentrations for 72 h. An MTT assay was conducted to test toxicity levels and calculate IC50 value (FIG. 12). The MTT assay revealed that overall, the free drug showed less toxicity toward the cells, because the hydrophobic free drugs failed to solubilize fully and reach the cells, whereas the NP-delivered ARVs were able to enter cells in higher levels and be delivered to the mitochondria due to the positive sur-face charge of the nanoparticle. The NPs and polymers themselves were previously shown not to cause cellular toxicity, so it was the delivery of the ARVs to the mitochondria that can have caused increased mitochondrial dysfunction and oxidative stress, making the drugs' toxic side effects more visible. Thus, it becomes evident that when ARVs can be delivered with a NP system to hard-to-reach viral reservoirs such as the brain. A combination therapeutic approach needs to be included to reduce both the ART- and virus-induced toxic effects such as inflammation and oxidative stress. To measure the effects of free drug and T-ARV-NPs on microglia, mitochondrial respiration was assessed and basal respiration, maximal respiration, coupling efficiency, and spare respiratory capacity were quantified using the Seahorse XF^{e}96 Analyzer. ARVs are known to cause oxidative stress and inflammation, which when compounded with the ROS and inflammation levels an HIV-positive patient already experiences, can lead to tissue damage and neurological effects. The effect of ART on ROS generation and inflammatory marker IL-1β was evaluated in microglia cells. Treatment with ARVs or ARV-loaded NPs (1 μM of free ARVs or NPs with respect to the loaded drug) increased ROS significantly for the majority of drugs tested, including saquinavir, efavirenz, darunavir, dolutegravir, and elvitegravir (FIG. 3A, FIG. 13). Similarly, levels of IL-1β, a marker for inflammation, were seen to increase significantly after treatment with NP-encapsulated ARVs, but not after treatment with the free ARVs (FIG. 3B). Previous studies demonstrated that the empty NPs do not cause any changes in ROS or inflammation. This indicated that the drugs encapsulated in the NPs are effectively targeted to the mitochondria and in-creased inflammation significantly. Despite the ROS and inflammation generated by the ART-NPs due to their ability to delivery drugs intracellularly and to the mitochondria, the ART-NPs still remain a more reasonable treatment option over free ARTs, particularly for treatment of HIV in the brain, as the free ARTs have limited ability to cross the BBB to re-duce/eliminate CNS HIV reservoirs. As much of the ART-NPs' toxicity is due to higher rates of intracellular drug delivery as compared to the relatively insoluble and hydrophobic free ARTs, this can allow for lower doses of ART-NPs to be used compared to the free ARTs. The majority of HIV-positive patients use drugs of abuse and are at higher risk of HAND. Two drugs of abuse, cocaine and meth, were used to understand how these drugs alter mitochondrial functions in microglia cells. Seahorse MitoStress assay data indicated that both cocaine and meth at 500 μM substantially decreased indicators of mitochondrial health, including basal and maximal respiration as well as ATP production (FIGS. 3C, 3D). The effects of cocaine and meth on human astrocytes were also followed by MitoStress assay under similar conditions and the data are presented in FIG. 14. These data showed that the damage to mitochondrial respiration and functions caused by drugs of abuse can worsen the effects of any ROS and inflammation caused by treatment with antiretroviral agents in HIV-positive patients. To tackle this issue, combination therapy of BBB-penetrating, mitochondria-targeted nanoparticles loaded with a prodrug of aspirin, Oc-[G2]-(Asp)₄, an anti-inflammatory agent, and CoQ₁₀, an antioxidant, can be used to supplement T-ARV-NP treatment in the HIV-positive patient which decrease inflammation and oxidative stress.

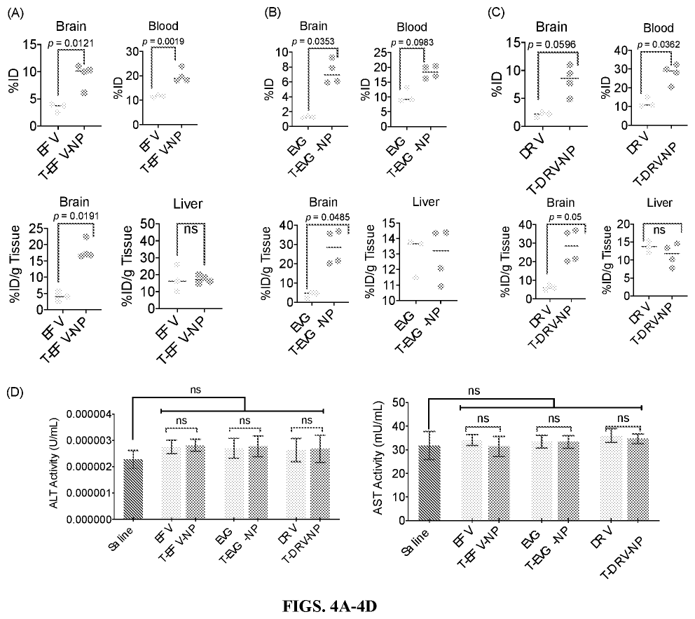

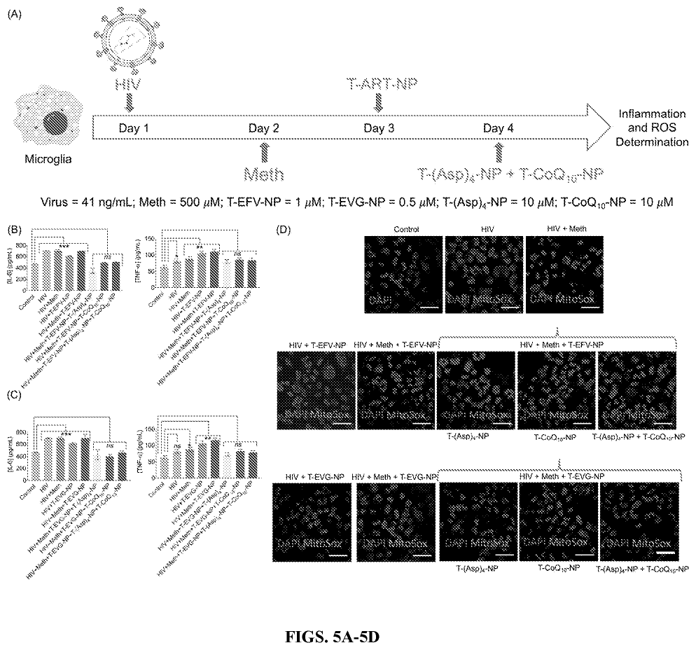

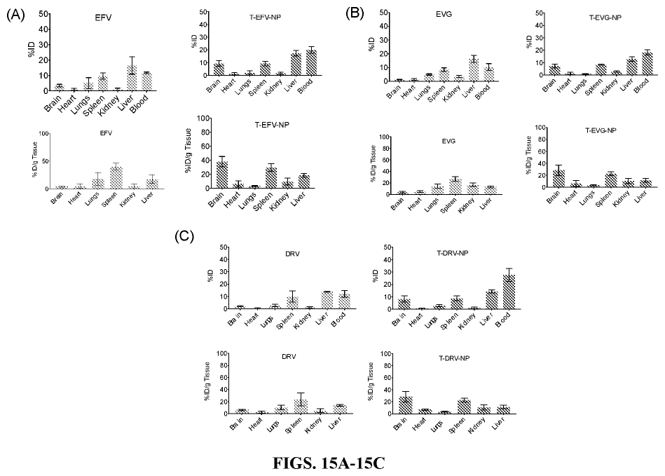

In Vivo Distribution of T-ARV-NPs. BALB/c albino mice were used to understand the distribution properties of T-ARV-NPs after intravenous administration. The animals were divided into the following seven groups: saline, efavirenz, darunavir, elvitegravir, T-EFV-NP, T-DRV-NP, and T-EVG-NP. The dose of NP was 40 mg/kg with respect to the drug. After 24 h, around 200 μL of blood was collected in heparinized tubes via cardiac puncture. The collected blood was centrifuged to isolate blood plasma. The animals were sacrificed and the major organs were harvested, weighed, and digested. The biodistribution was followed by running the samples through HPLC to calculate the percentage of NP in the organs and blood. This study indicated that the targeted, ARV-loaded NPs were able to cross the blood-brain barrier in significantly higher amounts than the free drug (FIGS. 4A, 4B, and 4C top). This is a crucial fac-tor in ensuring enough of the drug is delivered across the BBB to effectively treat HIV-infected cells. The drugs were also found to circulate significantly more amounts in the blood when delivered with the NPs (FIG. 4A, 4B, 4C top) indicating higher possibility of further accumulation in the brain as the time progresses. Further, it was observed that delivery using the NP system did not cause any significant increase in the liver accumulation of the ARVs compared with the free form (FIGS. 4A, 4B, 4C bottom). The whole distribution pattern by measuring % ID and % ID/g of tissue are presented in FIG. 15. As a whole, there was higher biodistribution and accumulation of the NP delivered ARVs as compared to the free ARVs, indicating the nanoparticles are improving the drugs' half-lives in the blood and as well as in the brain. In addition, there was no significant hepatotoxicity due to both the free ARVs and T-ARV-NPs, as shown by normal activity of the liver enzymes alanine aminotransferase and aspartate aminotransferase (FIG. 4D).

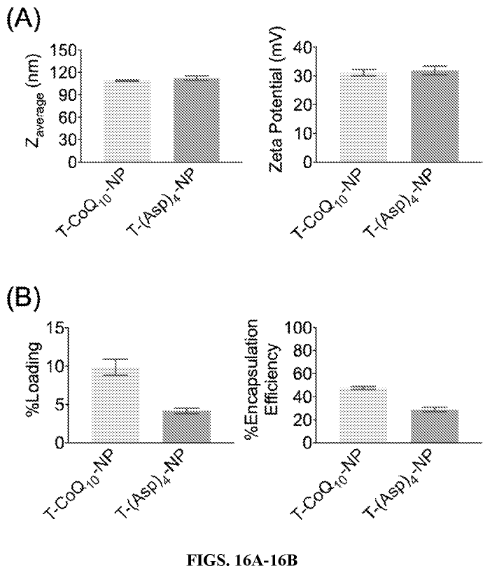

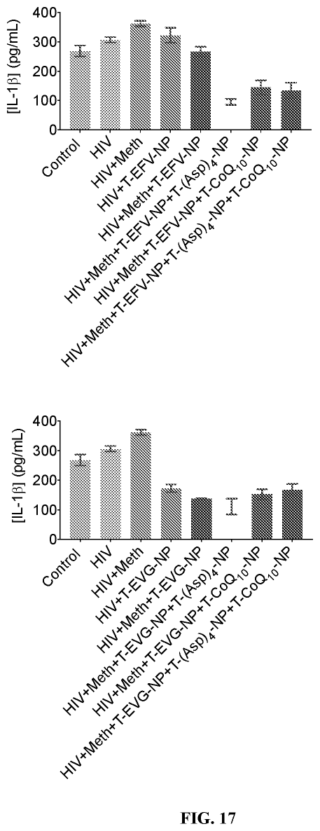

HIV-induced Inflammation and ROS are Reversed by T-(Asp)₄-NP and T-CoQ₁₀-NP Formulation. To mimic the treatment of HIV in a patient with a history of drug abuse, a sequence treatment was conducted over the course of 1 week which included microglia exposure to HIV and meth, followed by treatment consisting of EFV or EVG or the NPs and T-(Asp)₄/CoQ₁₀-NPs. A schematic of the experimental details is presented in FIG. 5A. Complete characterization of Oc-[G2]-(Asp)₄-loaded targeted NPs, T-(Asp)₄-NPs and CoQ₁₀-loaded NPs, T-CoQ₁₀-NPs are provided in FIG. 16. Afterward, the media was used to determine the level of the inflammatory markers IL-1β, IL-6, and TNF-α. The findings indicated the impact of HIV on the inflammatory cytokines and ROS levels of human microglia cells. HIV enhanced the levels of IL-1β, IL-6, and TNF-α. Abuse drug meth was also included to enhance the effects further. The cells were further treated T-EFV-NP or T-EVG-NP and then treated with T-CoQ₁₀-NP, T-(Asp)₄-NP, or their combination. Analyses of inflammatory responses by performing ELISA indicated that the combination of T-(Asp)₄-NP and T-CoQ₁₀-NPs was able to reduce the inflammatory signals caused by HIV as well as the enhanced levels caused by meth (FIGS. 5B and C, FIG. 17). Similar effects were found when mitochondrial ROS was assessed using Mito-SOX (FIG. 5D).

Protection of Microglia from HIV- and Meth-induced Toxic Effects by T-CoQ₁₀/(Asp)₄-NP-Nourished Astrocytes. Astrocytes play crucial roles to protect neurons by providing extensive support in terms of structure, metabolic processes during neurodegenerative processes. Relatively more resistance of astrocytes than neurons towards ROS, mitochondrial dysfunctions, and other environmental damages give the astrocytes roles to act as natural protectants of neurons. But, when HIV infection and drug abuse trigger mitochondrial dysfunctions in astrocyte population that in succession affect neurons. Thus, if astrocytes can be protected from mitochondrial dysfunctions, there is a possibility of neuronal survival. The targeted NPs which in this work not only crosses the BBB but also localizes in the astrocytes; and this NP system targets mitochondria with high efficacy. The overall goal in the experiment was to study whether this BBB penetrating NP with abilities to incorporate and deliver mitochondria acting natural anti-oxidant CoQ₁₀ and prodrug of aspirin to astrocytes to achieve astrocyte mediated neuronal protection against HAND and drug abuse conditions.

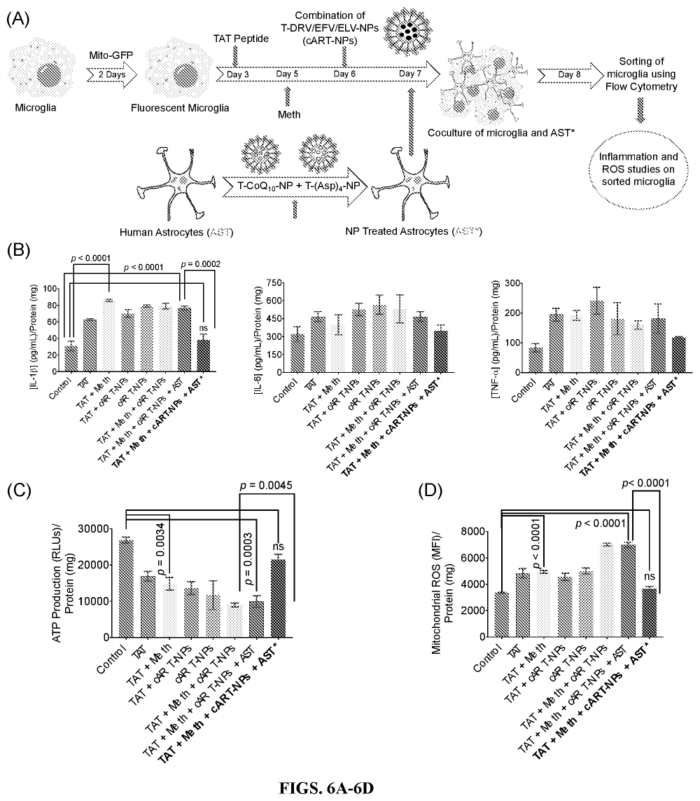

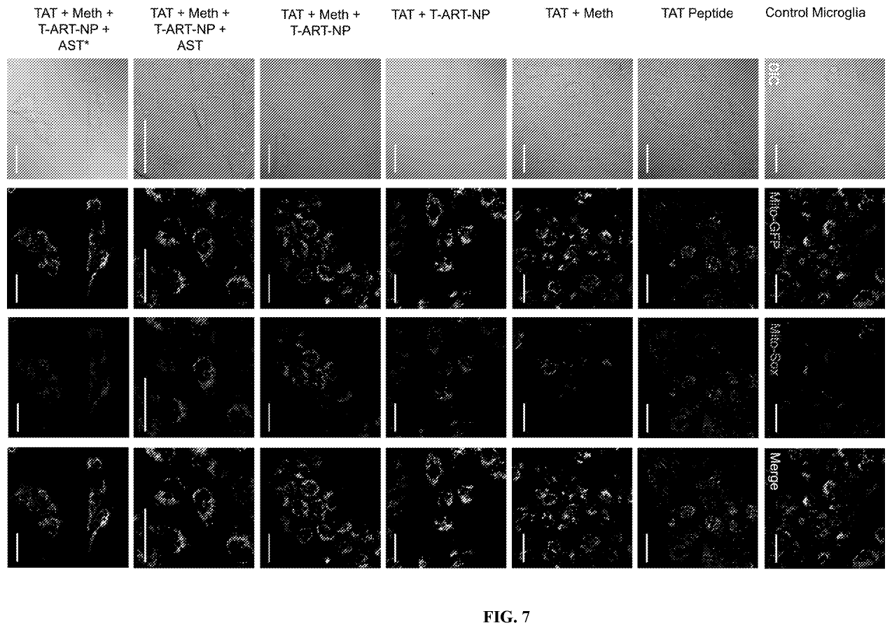

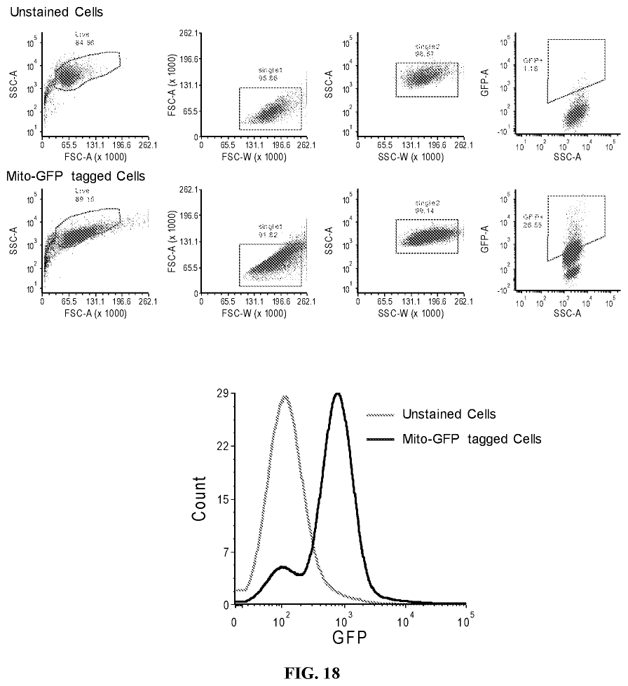

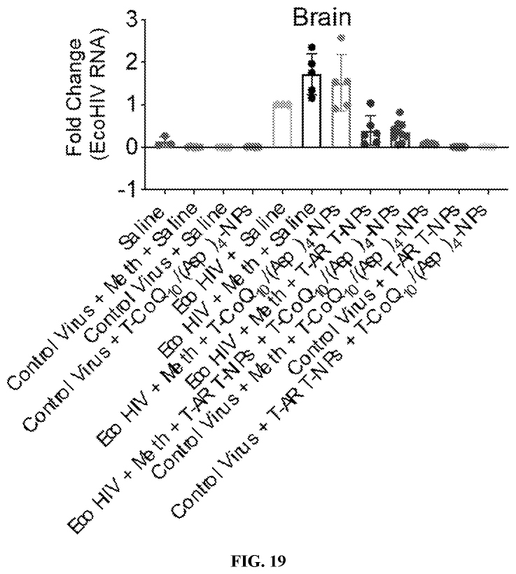

The study aimed at simulating HIV infection and drug use in patients with the goal of examining astrocyte-mediated protection of microglia by reducing inflammation and oxidative stress in HIV-infected microglia through co-culturing them with astrocytes treated with the T-CoQ₁₀/(Asp)₄-NPs, rather than directly treating those microglia with the antioxidant and anti-inflammatory nanoparticles (FIG. 6A). On day 1, microglia were treated with Mito-GFP, a signaling protein that allows the mitochondria to fluoresce. On day 3, microglia were treated with 200 ng/mL of TAT peptide, to induce HIV infection-like conditions within the cells. On day 5, microglia were exposed to meth to mimic exposure of a patient's central nervous system (CNS) to drugs of abuse. On day 6, microglia were exposed to a combination of T-EFV-NP, T-EVG-NP, and T-DRV-NP, to simulate a combination antiretroviral therapy using the BBB-penetrating NPs. On day 7, the microglia were co-cultured with astrocytes. Certain experimental groups of these astrocytes were previously treated with the anti-inflammatory and antioxidant agents in nanoparticles, T-(Asp)₄-NP and T-CoQ₁₀-NP, in concentrations of 10 μM each. On day 8, microglia were sorted (FIG. 18) and examined for levels of inflammatory markers (FIG. 6B), ATP (FIG. 6C), and ROS (FIG. 6D). Microglia treated with TAT peptide, meth, and the T-ART-NPs showed a significant increase in the levels of the inflammatory markers IL-1β, IL-6, and TNF-α, and again, this increase occurred regardless of whether the microglia were kept alone or co-cultured with untreated astrocytes, indicating the inflammatory nature of the HIV infection, methamphetamine use, and T-ART-NP treatment (FIG. 6B). Compared to the control, microglia treated with TAT peptide, meth, and the T-ART-NPs showed a significant reduction in ATP production, both when these were kept alone or co-cultured with untreated astrocytes, suggesting the development of critical mitochondrial dysfunction (FIG. 6C). However, if those treated microglia were co-cultured with astrocytes (abbreviated as AST*) previously treated with the T-(Asp)₄-NPs and T-CoQ₁₀-NPs, there was a significant increase in ATP production, back up to levels close to the control's ATP production levels. Once again, compared to the microglia co-cultured with untreated astrocytes, a significant reduction in these inflammatory markers was seen once microglia were co-cultured with astrocytes previously treated with T-(Asp)₄-NPs and T-CoQ₁₀-NPs. The levels of inflammatory markers returned to the levels seen in untreated microglia, showing a strong protective and reparative effect of the healthy, treated astrocytes. Levels of mitochondrial superoxide, a reactive oxygen species, were seen to increase significantly upon exposure to HIV, meth, and the T-ART-NPs, with or without the co-culturing of untreated astrocytes (FIG. 6D). However, upon co-culturing with treated astrocytes, the level of superoxide decreased back down to the level of ROS in the control microglia. These results were further supported through the fluorescence imaging that shows the colocalization of mito-GFP and mito-SOX markers in the mitochondria of treated cells, followed by a decrease in fluorescence in microglia co-cultured with treated astrocytes (FIG. 7). These results paint a promising picture of therapies that take advantage of astrocyte-mediated protection and repair. Although the specific mechanism by which aspirin- and CoQ₁₀-treated astrocytes modulate a decrease in ROS and inflammation and mitochondrial rejuvenation in HIV-positive, meth-exposed microglia remains unknown, there is a clear improvement in these metrics upon the co-culturing with treated astrocytes. This indicates that the damage done by HIV infection, drugs, and subsequent therapies can be repaired through co-treatment in the CNS with the nanoparticle-based de livery of antioxidants and anti-inflammatory agents.

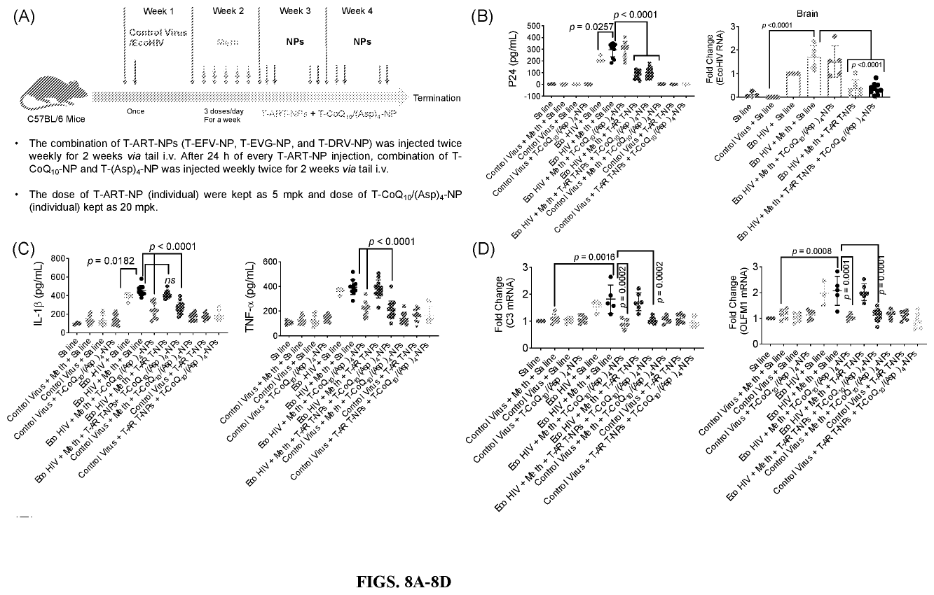

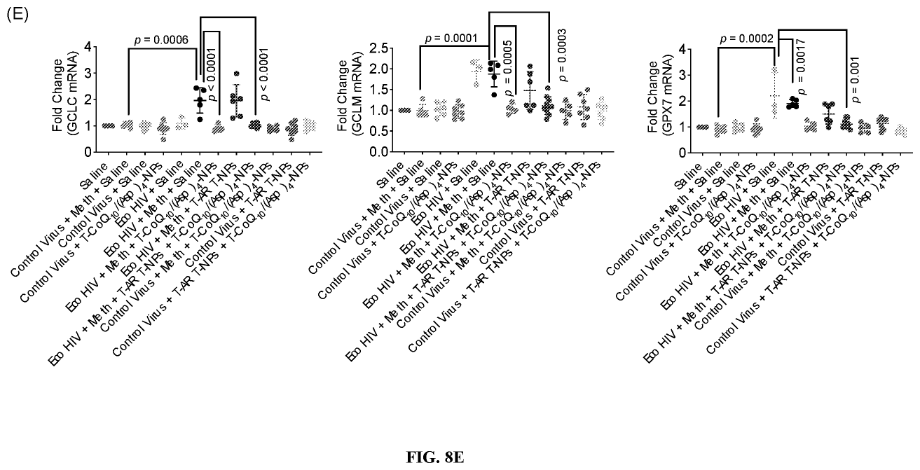

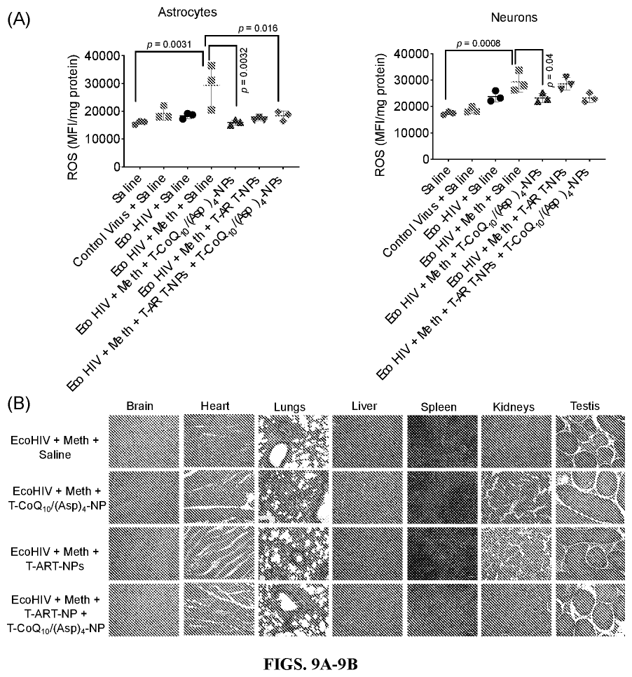

In Vivo Evaluation of T-ART-NPs and T-CoQ₁₀/(Asp)₄-NPs in EcoHIV-Infected Mice Under Methamphetamine Use Conditions. To evaluate the effectiveness of the combination of BBB penetrating, antiviral, antioxidant, and anti-inflammatory NPs in a living system, mice were infected with ecotropic HIV (EcoHIV) via tail i.v. and were subsequently exposed to meth to emulate patients with substance abuse issues. EcoHIV is a modified form of HIV with surface gp120 molecule removed so that the virus can only infect mice. A control virus pBMN-I-GFP was also injected, to serve as a control for EcoHIV which does not cause any toxicity or inflammation in the mice. After meth exposure, the mice were treated with a combination T-ART-NP (T-EVG-NP, T-EFV-NP, and T-DRV-NP), followed by treatment with a combination of T-CoQ₁₀-NP and T-(Asp)₄-NP. After the experiment was concluded, the mice were sacrificed for ex vivo analyses. A detailed timeline and description of this experiment are represented in FIG. 8A.

First, levels of HIV viral protein p24 were evaluated from blood plasma and brain tissue lysate through ELISA and RT-PCR, respectively (FIG. 8B, FIG. 19). In EcoHIV positive mice exposed to meth, the levels of p24 antigen were elevated, except for those treatment groups which received T-ART-NP; these groups showed a reduction in p24 levels (FIG. 8B). This indicates that the T-ART-NPs are highly effective at treating HIV. Further, anti-inflammatory cytokines IL-1β and TNF-α were evaluated in blood plasma through ELISA and these levels were increased in both EcoHIV infected as well as EcoHIV and meth exposed mice (FIG. 8C). The inflammation was decreased with the treatment of T-CoQ₁₀/(Asp)₄-NPs alone and in combination with T-ART-NPs (FIG. 8C). RT-PCR was used to evaluate mRNA levels of neuroinflammation markers C3 and OLFM1 in brain lysates (FIG. 8D). EcoHIV infected mice showed a rise in neuroinflammation which was decreased after the treatment of T-CoQ₁₀/(Asp)₄-NPs alone and in combination with T-ART-NPs (FIG. 8D). Glutamate-cysteine ligase catalytic subunit (GCLC) and glutamate-cysteine ligase modifier subunit (GCLM) are the enzymes that are involved in glutathione synthases to control the elevated ROS level. Glutathione peroxidase 7 (GPX7) is a member of glutathione peroxidase family which regulates ROS. RT-PCR analyses measured the mRNA levels of the ROS markers GCLC, GCLM, and GPX7 (FIG. 8E). The data revealed that mice infected with EcoHIV and exposed to meth had high levels of ROS and treatment with T-ART-NPs did not cause any changes in the ROS. When treated with the T-CoQ₁₀/(Asp)₄-NPs there was a reduction in these levels, indicating the NPs are effective at reducing oxidative stress caused by substance abuse along with EcoHIV infection (FIG. 8E). Overall, the combination of T-ART-NPs and T-CoQ₁₀/(Asp)₄-NPs is able control the neuroinflammation and the ROS levels in the brain caused by EcoHIV infection.

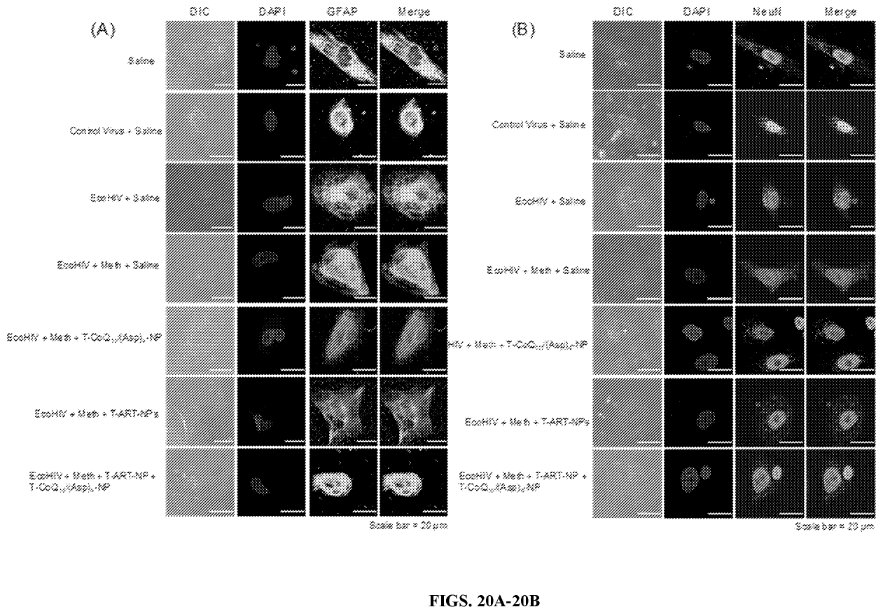

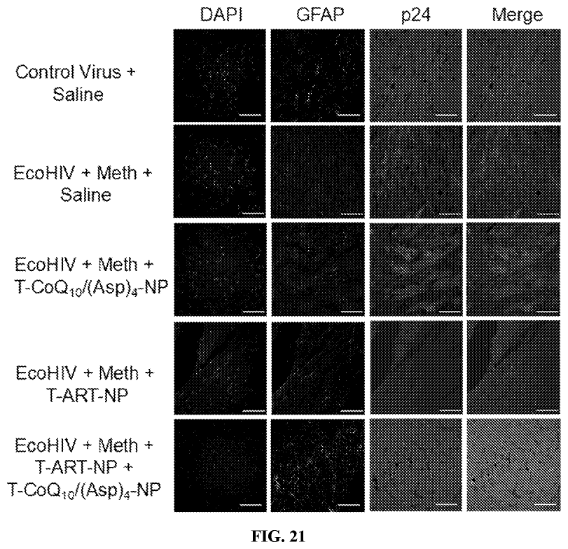

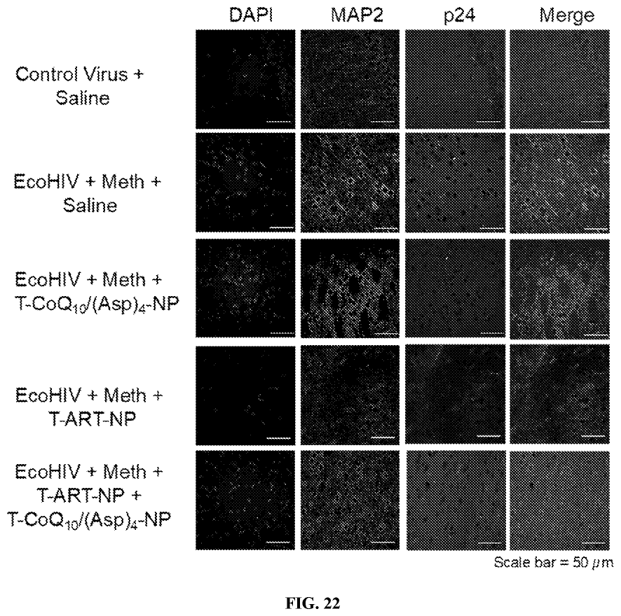

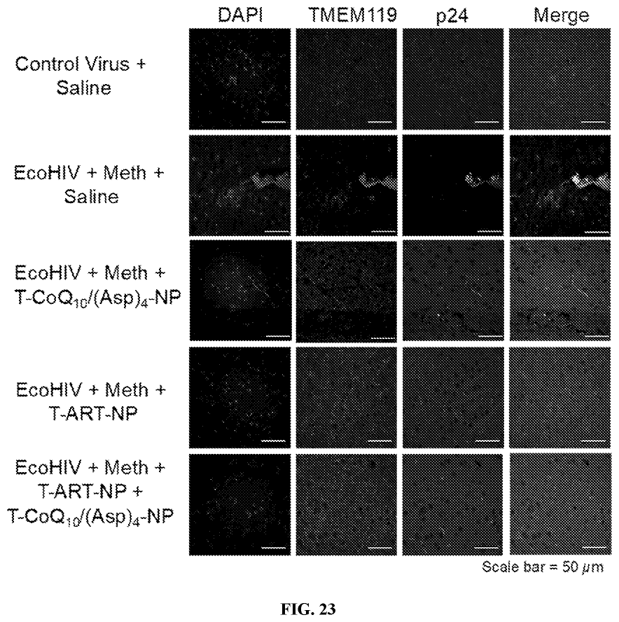

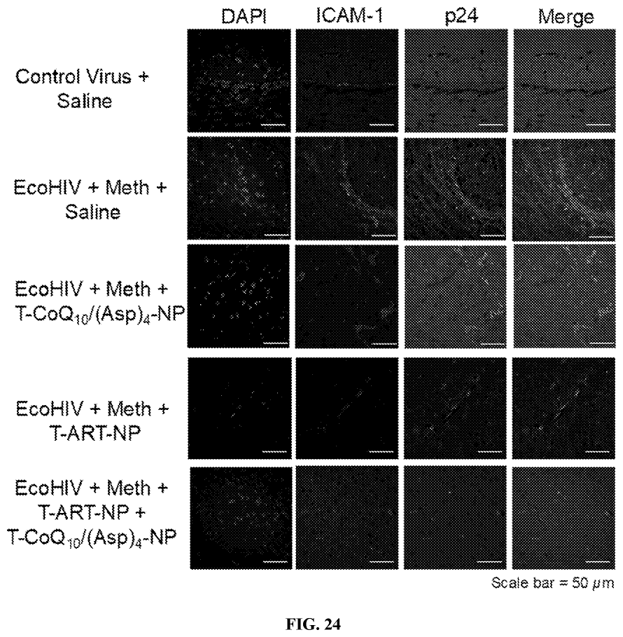

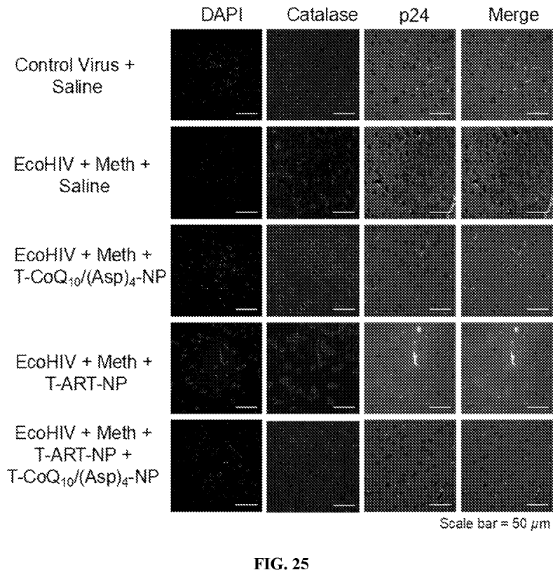

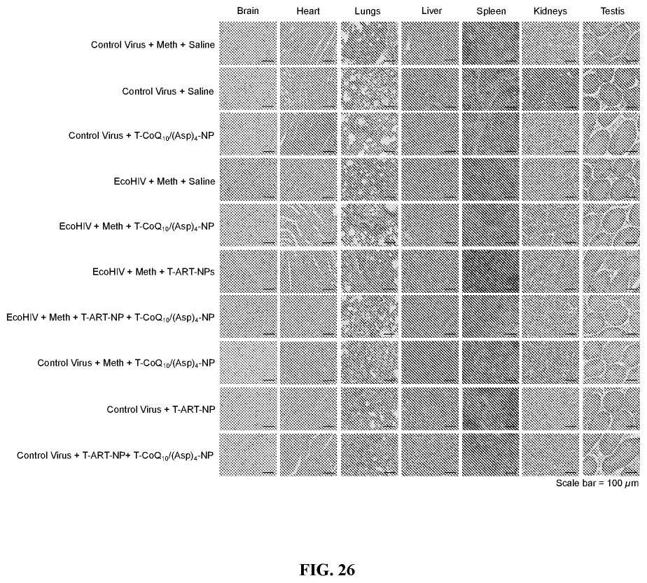

The ROS levels were evaluated in glial cells and neurons that were isolated from freshly harvested brain samples and grown in their respective selective media. The ROS level was increased in astrocytes and neurons from EcoHIV infected mice and reduced in the mice that were treated with the T-CoQ₁₀/(Asp)₄-NPs (FIG. 9A). The isolated cells were characterized with GFAP and NeuN marker for astrocytes and neurons, respectively (FIG. 20). Immunofluorescence was performed on brain tissue samples to study the site of accumulation of the viral particle. The samples were stained for p24 viral protein along with GFAP for astrocytes (FIG. 21), MAP2 for neurons (FIG. 22), TMEM119 for microglia (FIG. 23), ICAM-1 for signaling factor involved in neuroinflammation (FIG. 24), catalase for ROS marker (FIG. 25). These data indicated that treatment using the T-ART-NPs and T-CoQ₁₀/(Asp)₄-NPs showed reduction in HIV infection in the brain, levels of inflammation, and oxidative stress (FIGS. 19-23). H&E staining was conducted to observe tissue damage in all organs, which did not reveal any structural toxicity (FIG. 9B, FIG. 26). One of the significant aspects of this study is the ability of the NPs to effectively accumulate in the brain. This property leads to an increased bioavailability of the ARTs in the viral reservoirs of the brain which is difficult to achieve with free form of the ARTs. With this platform various classes of antiviral drugs were delivered to the brain. In addition, the combination of these ART loaded NPs along with T-CoQ10/(Asp)4-NPs were also able to lower the neuroinflammation and ROS levels caused by EcoHIV and drug of abuse. Taken together, these studies revealed that combination therapy with the nanoparticles can result in a highly effective treatment regimen for the HIV infected population who are addicted to the substance of abuse.

This work combined a lipophilic, biodegradable, brain-accumulating, intracellular-targeted NP containing ARTs with NPs containing antioxidants and anti-inflammatory agents to treat HIV infection in a model of HIV infection and recreational drug use. This work is based on a combination of synergizing conceptual and technical innovations to gain an understanding of the effectiveness of combination therapy and future clinical translation. In particular, this work provides a platform to (a) obtain knowledge on the effects of brain-accumulating NPs containing ARTs, anti-oxidants, and anti-inflammatory agents on the HIV-infected population; (b) utilize the inherently hyperpolarized mitochondria of astrocytes and microglia to target these cell populations using the brain-penetrating NP system containing mitochondria-acting anti-oxidant and anti-inflammatory drugs; (c) deliver an anti-oxidant inside the mitochondrial lumen where dysfunctions and ROS are located; (d) simultaneously deliver an anti-inflammatory agent; and (e) develop the biodegradable NP used in this study from a single-step, controlled procedure that produces NPs with distinct properties. In synthesizing the nanoparticles, all chemical conjugations occur before NP formulation from the polymers, which minimizes variability. In addition, through an extensive study in a model of HIV infection and intravenous drug use, the combination nanoparticle treatment was shown to be effective in reducing HIV infection, inflammation, and oxidative stress. This approach is robust, simple in design, and scalable, making it well suited for clinical translation.

The nanoparticles (NPs) made from the polymer poly(lactic-co-glycolic acid) (PLGA)-block(b)-polyethylene glycol (PEG) functionalized with a terminal triphenylphosphonium (TPP) cation have shown promising physical characteristics which allow them to circulate efficiently in blood and pass the BBB to accumulate in the brain. In this study, recently developed antiretroviral (ARV)-loaded NPs are described, along with their characterization and loading efficacies, and cytotoxicity studies. Also, the efficacies of these NPs in tackling HIV are shown by presenting the results obtained from p24 ELISA assays and quantitative polymerase chain reaction (qPCR) after treating cells with Tat protein or ecotropic HIV (ecoHIV) or HIV-in in vitro and/or in vivo settings. These results indicate that the NPs loaded with antiretrovirals as well as the antioxidant Coenzyme Q10 and a prodrug of the anti-inflammatory agent aspirin, (Asp)4, are therapeutically effective at tackling various symptoms generated from Tat, EcoHIV, HIV and reducing associated oxidative stress and inflammation. This combination treatment can be particularly useful for patients who have elevated reactive oxygen species and inflammation due to the use of intravenous drugs such as meth or already on HIV treatment drugs. The therapeutic NP can serve as an effective treatment for all HIV-positive individuals or at-risk population for infection in the brain or people under HIV treatment which is generating significant ROS and inflammation in blood and brain.

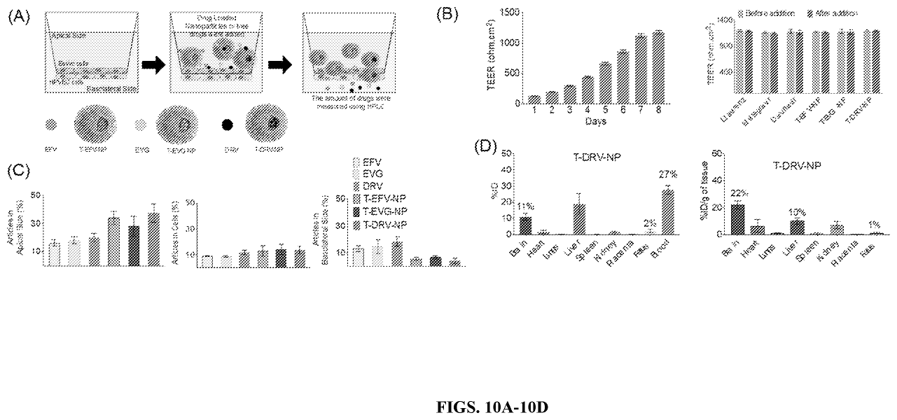

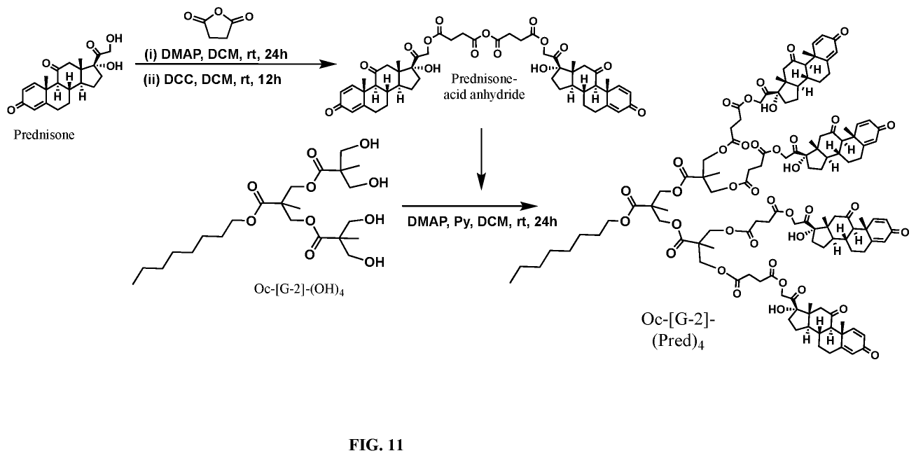

Nanoparticle Distribution Using In Vitro Placental Barrier Model. In pregnant women, the mother and fetus are separated by the placental barrier which is built by endothelial and epithelial cells along with connective tissue and allows for diffusion of different substances between the maternal and fetal circulatory systems. By building an in vitro placental barrier from human placental vascular endothelial cells (HPVEC) and placental epithelial cells (BeWo), we envisioned studying the fate of ARTs and ART-loaded nanoparticles at the maternal side of the barrier (FIG. 10A). Before the addition of articles, the monolayer integrity was checked by measuring trans-epithelial electrical resistance (TEER), indicating a value of >1000 Ω/cm² on day 8 (FIG. 10B). The TEER values confirmed that the addition of the articles did not damage the placental barrier (FIG. 10B). Apical basal media and the cell pellets were collected in eppendorf tubes and dissolved in 2 mL of methanol. As an internal standard, 10 μg/mL of respective ARTs were added to the collected media. This mixture was sonicated for 20 min followed by centrifugation at 5000 RPM for 10 min. From the precipitated debris, the supernatant was gently collected. Meanwhile, Strata C18-T columns were activated by passing 1 mL of methanol and water through the filter in sequence. The collected supernatant was passed through the activated column in order to get rid of remaining debris and impurities. The column was washed with 1-2 mL of 5% methanol in order to remove the impurities. The drugs from the column was collected in 2 mL of methanol and quantified using IPLC [efavirenz (27.3 min, 268 nm), darunavir (24.0 min, 268 nm), and elvitegravir (27.9 min, 268 nm)]. It was found that the free ARTs, EFV, EVG, and DRV, were able to cross the placental barrier in higher amounts compared to their respective nanoparticles (FIG. 10C). While some of the ART-loaded nanoparticles were taken up into the cells forming the barrier, the majority remained in the apical medium and very low amounts passed into the basal medium. The rate of passage was far lower for the ART-loaded nanoparticles compared to the free drugs, which is crucial in preventing drug-related toxicity to the fetus, which could potentially result in elevated oxidative stress, inflammation, and tissue damage.

Biodistribution of ARV-loaded NPs in Pregnant Mice. The distribution of the targeted T-DRV-NPs was carried out in pregnant female C57BL/6 mice after intravenous administration. Female mice of 10-12 weeks were housed with male mice to induce pregnancy. On the 14^{th} day following confirmation of copulation plug, the mice were administered with NPs. Saline-treated mice were used as a control. The animals were divided into two groups: saline (n=1), T-DRV-NP (n=2). The animals received saline treatment or T-ARV-NP treatment via tail i.v. The dose of NP was kept as 40 mg/kg with respect to DRV weight. After 24 h of injection, under anesthesia, around 400 μL of blood was collected via cardiac puncture and perfusion was carried out with 1×PBS for 10 min with a flow rate of 7 mL/min. After perfusion, the animals were sacrificed and the organs were harvested. The collected blood was centrifuged to collect blood plasma. The collected organs were weighed and homogenized using a dounce homogenizer and collected in 5 mL of methanol. To spike the peak, 20 μg/mL of the DRV was added to the crushed tissues and to the blood plasma. This mixture was sonicated for 20 min followed by centrifugation at 14,000 rpm for 10 min. From the precipitated debris, supernatant was gently collected. Meanwhile, Strata-X columns were activated by passing 1 mL of methanol and 1 mL of water through the filter in sequence. The collected supernatant from the tissue was passed through the activated column to remove remaining debris and impurities. The column was washed with 1 mL of 5% methanol to remove the impurities. The ART from the column was collected in 1.5 mL of methanol and quantified using HPLC (Wavelength: 24.0 min, 268 nm). The T-DRV-NPs were found to preferentially accumulate in the brain upon intravenous administration, demonstrating the BBB-penetrating nature of the nanoparticles and delivery of the ART to the brain. A significant amount of the NP remained circulating in the blood and also localized to the liver and kidney, but a high enough amount accumulated in the brain for the NP-based therapy for delivery of an effective dose of the ART. Another crucial finding was that the nanoparticle accumulation in the placenta and fetus was found to be very low to negligible. The inability of the NPs to penetrate the placental barrier is an important characteristic to ensure the NPs are able to safely treat HIV in pregnant women without adverse effects on the fetus (FIG. 10D).

### Synthesis of Oc-[G-2]-(Pred)₄

(i) Synthesis of prednisone mono-succinate acid: Prednisone (0.2 g, 0.558 mmol), DMAP (68.41 mg, 0.558 mmol) and succinic anhydride (55.83 g, 0.558 mmol) were dissolved in 60 mL of dichloromethane and left to react overnight under stirring at rt for 3 days. Then, the reaction mixture was quenched with 5 mL of water. Subsequently, the reaction mixture was diluted with 50 mL of dichloromethane and extracted with 10% aq. NaHSO₄ (3×20 mL) and brine. The organic phase was dried over MgSO₄ and the solvent was evaporated to dryness to obtain a white

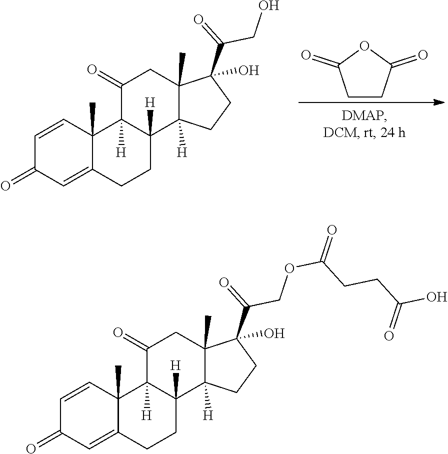

solid product. Yield 160 mg, 62% and second time 175 mg, 68%. ¹H NMR (CDCl₃, 400 MHz): δ PPM, 7.69 (d, 1H), 6.22 (d, 1H), 5.08 (d, H), 4.70 (d, 1H), 2.85 (d, 1H). 2.72 (m, 5H), 2.27-2.51 (m, 4H), 1.90-2.07 (m, 4H), 1.69 (m, 1H), 1.42 (s, 4H). 1.26 (m, 1H), 0.67 (s, 3H). ¹³C NMR (CDCl₃, 100 MHz): δ 208.96, 186.63, 171.73, 155.63, 127.47, 124.51, 89.56, 67.89, 60.15, 51.42, 49.58, 49.51, 42.43, 36.06, 34.93, 33.66, 32.23, 28.66, 28.52, 23.23, 18.73, 15.48.

(ii) Synthesis of prednisone acid anhydride: A suspension of monosuccinato-prednisone (200 mg, 0.436 mmol) in 15 mL of CH₂Cl₂ was prepared and a solution of 1,3-dicyclohexylcarbodiimide (DCC) (45 mg, 0.2183 mmol) in 5 mL of CH₂Cl₂ was added. The reaction mixture was stirred at room temperature for overnight. The urea DCC by product dicyclohexylurea (DCU) was filtered off in a glass filter and washed with a small amount of CH₂Cl₂. The solvent was evaporated and the resulting residue was taken up in EtOAc. Residual DCU was removed by filtering the resulting suspension through a glass filter. The filtrate was evaporated to give anhydride as white solid oil. Compound was used directly for next reaction. Yield 219 mg, 50%. ¹H NMR (CDCl₃, 400 MHz): δ PPM, 7.66 (d, 1H), 6.19 (d, 1H), 5.07 (d, 1H), 4.74 (d, 1H), 2.73-2.87 (m, 6H). 2.29-2.48 (m, 4H), 1.91-2.05 (m, 6H), 1.56-1.69 (m, 3H), 1.40 (s,

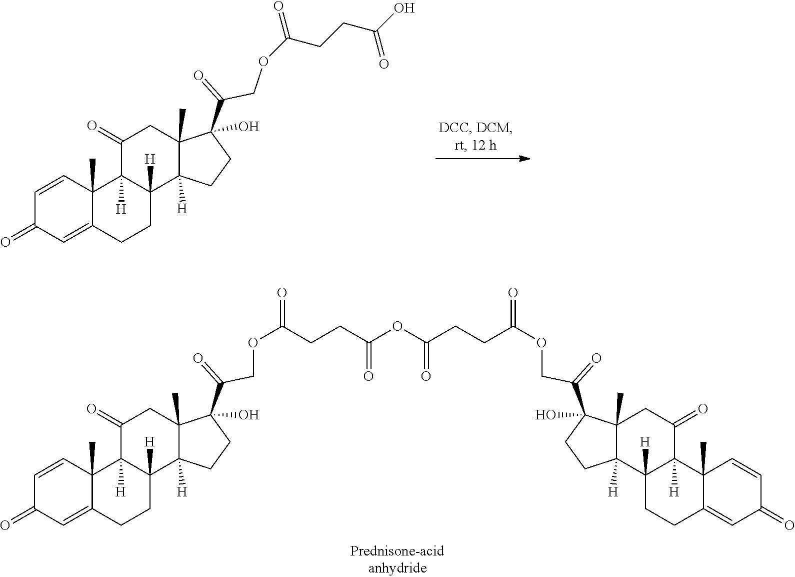

4H), 1.28 (m, 2H). 1.10 (m, 1H), 0.65 (s, 3H). ¹³C NMR (CDCl₃, 100 MHz): δ 190.35, 186.64, 171.20, 155.20, 127.41, 124.59, 113.29, 88.53, 68.03, 60.15, 51.47, 49.57, 42.36, 36.08, 33.66, 32.21, 30.18, 28.21, 24.80, 23.24, 18.78, 15.45.

(iii) Synthesis of Oc-[G-2]-(Pred)₄: Oc-[G-2]-(OH)₄ (19.54 mg, 0.046 mmol) and DMAP (3.45 mg, 0.028 mmol) were dissolved in of CH₂Cl₂ in a 50 mL round bottom flask. The anhydride of monosuccinato-prednisone (219 mg, 0.243 mmol) was added slowly. The solution was stirred at room temperature overnight. The reaction was quenched with 4 mL of water under vigorous stirring, followed by dilution with 50 mL of CH₂Cl₂ and the solution was washed with and 10% of Na₂CO₃ (3×20 mL) and brine (10 mL). The organic phase was dried with MgSO₄, filtered, and

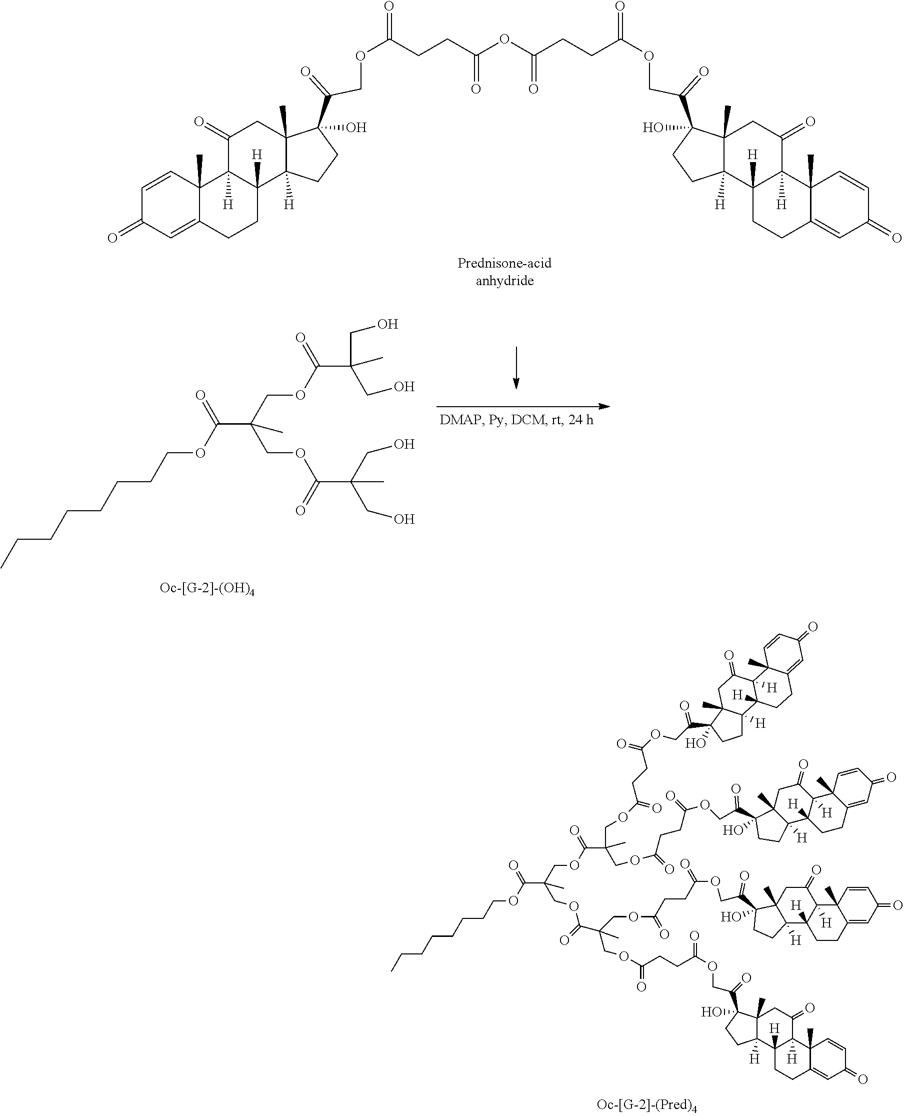

concentrated to get pasty mass as product. ¹H NMR (CDCl₃, 400 MHz): δ PPM, 7.66 (d, 3H), 6.14 (d, 3H), 6.03 (s, 3H), 5.02 (d, 3H), 4.70 (d, 3H), 4.18 (m, 6H), 2.86 (m, 3H). 2.67 (m, 12H), 2.25-2.49 (m, 14H), 1.66-1.99 (m, 18H), 1.53 (m, 8H), 1.39 (s, 12H), 1.06-1.23 (m, 18H), 0.61 (s, 9H). ¹³C NMR (CDCl₃, 100 MHz): δ 209.58, 205.09, 186.89, 175.20, 172.02, 167.91, 157.69, 156.26, 127.24, 124.25, 88.43, 68.24, 60.01, 51.40, 49.50, 49.23, 42.57, 36.08, 34.61, 33.58, 32.29, 28.84, 28.71, 25.46, 24.80, 23.24, 18.79, 18.70, 15.38.

### Materials and Methods

Materials: All chemicals were used without further purification unless otherwise noted. The polymer PLGA-b-PEG-TPP, 6-bromohexanoic acid, Oc-[G2]-(Asp)₄ were synthesized using previously published methods. Antiretroviral (ARV) drugs, saquinavir mesylate, atazanvir, tenofovir disproxil fumarate, indinavir sulfate salt hydrate, ritonavir, zidovudine, lopinavir, efavirenz, darunavir, nevirapine anhydrous, lamivudine, fosamprenavir calcium, amprenavir, dolutegravir, elvitegravir, cobicistat, abacavir sulfate, emtricitabine, raltegravir potassium, maraviroc, enfuvirtide acetate, delavirdine mesylate, bictegravir, didanosine, zalcitabine, and stavudine were purchased from Sigma, AmBeed, A ChemBlock, Selectchem, and CarboSynth. CoQ₁₀ and 2′,7′-dichlorofluorescin diacetate (DCFDA) was purchased from Sigma Aldrich. XFe96 FluxPaks (SKU 102416-100) were purchased from Agilent Seahorse. NucBlue® live cell stain (Cat. No. R37605), CellLight™ mitochondria-GFP was purchased from thermos Fischer Scientifics. Astrocytes basal media and astrocytes SingleQuots™ Kit growth factors were purchased as a single kit (CC 3186) from Lonza. Pierce® Bicinchoninic acid (BCA) protein assay kit (Cat No. 23225) was procured from ThermoFisher Scientific. Regenerative cellulose membrane Amicon Ultra Centrifugal 100 kDa filters were purchased from Merck Millipore Ltd. Copper grids for transmission electron microscopy (TEM) were purchased from Electron Microscopy Sciences. TAT peptide (Cat. No. 2222) was procured from NIH. Human IL-1p (Cat. No. 557953), IL-6 (Cat. No. 555220) and TNFα (Cat. No. 555212) kits were purchased from BD Biosciences. Elisa kit for IL-2 (Cat no. 0801200) was purchased from ZeptoMetrix Corp. Methamphetamine (Cat. No. M8750) and cocaine (Cat. No. C5776) were purchased from Sigma Aldrich. The HIV Clade B (HIV-1 Ba-L, Cat. No. 510) viral particles were procured from NIH AIDS Reagent Program. Alanine transaminase colorimetric activity assay kit (Cat. No. 700260) was purchased from Cayman chemicals. Aspartate Aminotransferase Activity Assay Kit (Cat. No. Cat. No. MAK055-1KT) was purchased from Millipore Sigma. Strata-X™ columns (8B-S100-UBJ) were purchased from Phenomenex. Neurobasal media (cat #12348017) was purchased from Thermo Fischer scientific along with supplements. DNase-I (cat #04536282001) was purchased from was purchased from Millipore Sigma. Poly-D-Lysine (cat #A3890401) was purchased from ThermoFisher scientific. NeuN (ab177487), GFAP (ab4674), TMEM119 (ab209064), ICAM1 (ab179707), catalase (ab52477) primary antibodies were purchased from Abcam. MAP2 (D5G1) was purchased from Cell Signaling Technology. P24 primary antibody (ARP530) was procured from HIV reagent program. The secondary anti-chicken (ab6875), and anti-rabbit (ab150080) were purchased from Abcam. Goat anti-human secondary antibody (A11013) was purchased from Invitrogen. Elisa kits for IL1-β (cat #559603) and TNFα (cat #558534) were purchased from BD sciences. HIV-1 P24 antigen Elisa 2.0 kit (cat #0801002) was purchased from Zeptometrix. The chimeric HIV-NDK (abbreviated as EcoHIV) generously gifted by Dr. David Volsky, Icahn School of Medicine at Mount Sinai. A retroviral vector pBMN-I-GFP procured from Addgene (plasmid #1736).

Instruments: Nanopure water purification system (18.2 MΩ, Millipore Milli-Q Biocel). Slide-A-Lyzer MINI dialysis tubes were obtained from Thermo Scientific. High-performance liquid chromatography (HPLC) experiments were conducted using an Agilent 1200 LC series instrument equipped with automated injector, and UV and fluorescence detectors. Dynamic light scattering (DLS) measurements were performed on a Malvern Zetasizer Nano ZS system. Cells were counted using a Countess® automated cell counter (Invitrogen Life Technology). Plate reader analyses were performed on a Bio-Tek Synergy HT microplate reader. TEM images were acquired using a JEOL JEM-1400 equipped with a Gatan Orius SC 200D CCD digital camera. Mitochondrial bioenergetics assays were performed on XFe96 Extracellular Flux Analyzers (Agilent Seahorse Biosciences). Confocal microscopy images were obtained using an Olympus FluoView FV3000. Microglia cells were sorted using an LSR-Fortessa-HTS instrument at the core facility of University of Miami. Real-Time PCR (RT-PCR) studies were carried out using CFX Connect System from BIO-RAD. Reverse Transcription Supermix for RT-qPCR kit was obtained from Bio-Rad. Real time PCR reaction was carried out using SsoAdvanced Universal SYBR® Green Supermix in 20 μL reaction. β-actin was used as an internal control. Data were analyzed according to the comparative Ct value and expressed as fold change 2-ΔΔCT. HIV was assessed using the following primers and probe: NDKgag_F 5′-GACATAAGACAGGGACCAAAGG-3′ (SEQ ID NO: 1); NDKgag_R 5′-CTGGGTTTGCATTTTGGACC-3′ (SEQ ID NO: 2). The primer sequence for β-actin gene was: Forward 5′GCATCCTCACCCTGAAGTAC3′ (SEQ ID NO: 3) and reverse 5′GATAGCACAGCCTGGATAGC3′ (SEQ ID NO: 4). GAPDH (4326317E), C3 (Mm00437859_g1) and OLFM1 (Mm00444666_m1) was purchased from Life Technologies. The primers for ROS marker purchased from Integrated DNA Technologies (IDT). The primer sequence for GCLC gene: Forward 5′ACACCTGGATGATGCCAACGAG3′ (SEQ ID NO: 5) and reverse 5′CCTCCATTGGTCGGΔΔCTCTAC3′ (SEQ ID NO: 6). The primer sequence for GCLM gene: Forward 5′TCCTGCTGTGTGATGCCACCAG3′ (SEQ ID NO: 7) and reverse 5′GCTTCCTGGAΔΔCTTGCCTCAG3′ (SEQ ID NO: 8). The primer sequence for GPX7 gene: Forward 5′CGACTTCAAGGCGGTCAACATC3′ (SEQ ID NO: 9) and reverse 5′AAGGCTCGGTAGTTCTGGTCTG3′ (SEQ ID NO: 10).

Cells and Culture Condition: Human microglia cells were purchased from ATCC and grown in Dulbecco's modified eagle's medium (DMEM) along with 10% fetal bovine serum (FBS). Cell cultures were maintained in a humidified cell culture incubator at 37° C. and with 5% CO₂. Normal human astrocytes (nHA) were purchased from Lonza. These cells were cultured in astrocyte basal media (ABM) supplemented with astrocytes SingleQuots™ Kit growth factors composed of FBA, L-glutamine, gentamycin, and ascorbic acid. Cell cultures were maintained in a humidified cell culture incubator at 37° C. and with 5% CO₂.

The isolated astrocytes were grown in glial media (Dulbecco's modified eagle's medium along with 10% fetal bovine serum (FBS). The isolated neuronal cells were grown in Neurobasal media with 10% FBS and 2% of B27 supplement. Cell cultures were maintained in a humidified cell culture incubator at 37° C. and with 5% C02.

Nanoparticle Synthesis: A library of targeted NPs was first prepared for preliminary testing using 5 mg/mL of the targeted polymer PLGA-b-PEG-TPP, 10% TPP-(CH₂)₅—COOH with respect to the polymer, and 10% of each ART (with respect to the polymer) taken from a 20 mg/mL stock solution in DMSO. The solutions were mixed in acetonitrile and made with a total volume of 1 mL, and added slowly and dropwise to water being stirred at 900 rpm. The solution was stirred for 2 h, then filtered using Amicon filtration (100 MWCO) at 2800 RPM. Initial DLS measurements of size and zeta potential were taken and the solutions were stored at 4° C. HPLC was used along with standards of the ARTs to see which of the ARTs showed signs of loading into the NPs. This was indicated by a presence of a peak occurring at the same elution time as the peaks for the ART standards. The HPLC elution time and max values were noted for the nine drugs that showed signs of loading: saquinavir (16.8 min, 268 nm), efavirenz (27.3 min, 268 nm), darunavir (24.0 min, 268 nm), dolutegravir (23.0 min, 268 nm), elvitegravir (27.9 minutes, 268 nm), raltegravir (22.8 min, 268 nm), delavirdine (20.6 min, 268 nm), bictegravir (23.3 min, 268 nm), and stavudine (13.3 min, 268 nm).

ART Feed Testing: For the ARTs that showed signs of loading, NPs were next prepared with varying feeds of the selected ARTs. NPs were prepared using 5 mg/mL of the targeted polymer PLGA-b-PEG-TPP, 10% TPP-(CH₂)₅—COOH, and ARTs at a feed of either 0.5 mg/mL (10%), 1 mg/mL (20%), 1.5 mg/mL (30%), 2 mg/mL (40%), or 2.5 mg/mL (50%). The NPs were produced and purified using the same process as above. HPLC was used to determine percent loading (% L) and percent encapsulation efficiency (% EE) for the various feeds of each ART into the targeted T-ART-NPs.

Release Studies: Release studies were conducted to test the release of ARTs loaded in the ART-NPs. 20 μL of 5 mg/mL nanoparticle solution (in water) was diluted to 200 μL using water. These solutions were then put into dialysis chambers and placed into phosphate buffered saline with pH 7.4. Samples were taken out at various time points up to 72 h. The solution remaining in the dialysis chambers was diluted in acetonitrile, and HPLC was used to test the amount of drug remaining in the solution. The HPLC elution time and λₘₐₓ values were noted for the three drugs that showed good loading: efavirenz (27.3 min, 268 nm), darunavir (24.0 min, 268 nm), and elvitegravir (27.9 min, 268 nm).

Cell Viability Assays: The cytotoxicity of Efavirenz, Elvitegravir, Darunavir, and their nanoformulations T-EFV-NP, T-EVG-NP, and T-DRV-NP were tested in microglia cells using the MTT assay. The cells were plated (3000 cells/well) in a 96-well plate and allowed to grow overnight. The media was changed and increasing concentrations of each article were added. The media was aspirated and fresh media was added and further incubated for an additional 48 h. After the given incubation time, 20 μL/well MTT was added (5 mg/mL stock in PBS) and incubated for 5 h in order for MTT to be reduced to purple formazan. The media was removed and the cells were lysed with 100 μL of DMSO. In order to homogenize the formazan solution, the plates were subjected to 10 min of gentle shaking and the absorbance was read at 550 nm with a background reading at 800 nm with a plate reader. Control values were set to 100% of cell viability. Cytotoxicity data was fitted to a sigmoidal curve and a three-parameter logistic model was used to calculate the IC₅₀, which is the concentration of articles causing 50% inhibition in comparison to untreated controls. The mean IC₅₀ is the concentration of agent that reduces cell growth by 50% under the experimental conditions and is the average from at least three independent measurements that were reproducible and statistically significant. These analyses were performed with GraphPad Prism.

ROS Production Assay in Microglia Cells: The ROS generation in the microglia cells were determined using DCFDA assay. Microglia cells were plated in the white wall coated 96-well plate with density of 20,000 cells per well and grown for 16 h. The SQV, EFV, DRV, DTG, EVG and their nanoformulations were added to the cells at concentration of 1 μM with respect to the ARTs for 24 h. After 24 h, the media was collected and used to determine IL-1β, an inflammation maker using commercially available kit. The DCFDA solution was made in media at the concentration of 100 μM. 100 μL of this solution was added to each well and cells were incubated for 30 min. The cells were washed with 1×PBS for 3 times and fluorescence of DCF was recorded at 495/528 nm (ex/em) using a BioTek Plate Reader. The obtained values were normalized using BCA assay.

ELISA in Microglia Cells: Using the supernatant from the assay, ELISA was carried out according to the manufacturer's instructions. Briefly, a 96-well plate was coated with 100 μL of capture antibody overnight at 4° C. The plate was washed with washing buffer, tapped on a paper towel and blocked with blocking buffer for 1 h at room temperature. Samples and standards were added to their respective wells and incubated for 2 h at room temperature. The plate was washed with washing buffer and tapped on paper towel 5 times. 100 μL of working detector solution was added to the well and incubated for 1 h at room temperature. After extensive washing, 100 μL of substrate solution was added to each well and incubated for 30 minutes at room temperature in the dark. Then, 50 μL of stop solution was added to each well and absorbance was recorded at 450 nm using a plate reader. The obtained values were normalized using BCA assay.

Mitostress Assay: Different parameters of mitochondrial respiration such as basal respiration, maximal respiration, and ATP production were investigated using Seahorse XFe96 Analyzer. One day prior to the assay, XF sensor cartridges were hydrated using 200 μL of XF calibrant buffer and kept at 37° C. incubator without CO₂ overnight. Cells were plated at a density of 20,000 cells per well in 50 μL DMEM media (with 10% FBS) and the plate was kept 1 h at room temperature followed by incubation at 37° C. with 5% CO₂ for 3 h. Finally, 130 μL of fresh media was added to have total 180 μL per well and incubated for 16 h. Cells were treated with Cocaine and Meth from 0 to 500 μM for 24 h. Before conducting the Mitostress assay, Seahorse media (XF Assay Medium Modified DMEM) was reconstituted with glucose (1.8 mg/mL), sodium pyruvate (1%) and L-glutamine (1%) and adjusted for pH 7.4 by using 0.1 N NaOH. The cells were washed thrice with freshly prepared seahorse medium and incubated at 37° C. in non-CO₂ incubator for 1 h. Meanwhile, cartridge ports were added with various inhibitors. The stocks of oligomycin (10 μM), FCCP (10 μM) and antimycin-A/rotenone mixture (10 μM each) were made in seahorse media. The port A was filled with 20 μL of oligomycin, port B with 22 μL of FCCP and port C with 25 μL of antimycin A/rotenone to have a final concentration of 1 μM in each well. The cartridge was calibrated for pH and 02. After calibration, the experiment plate was run where 3 measurements were recorded for basal OCR and after addition of each reagent. The media was aspirated and 20 μL of RIPA buffer was added to each well and incubated for 10 mins at 37° C. Further BCA assay was performed to obtain protein normalized OCR values.

HIV-1 Propagation and Expansion in Peripheral Blood Mononuclear Cells (PBMCs): PBMCs were separated from blood samples by using the density gradient method previously reported.³⁻⁴ Isolated PBMCs were incubated at the density of 5′10⁷ cells in 30 mL of RPMI for overnight. Next add 3 μg/mL of PHA was added to activate the cells. After 72 h, cells were centrifuged and the pellet was resuspended in 10 mL of RPMI containing 2 μg/ml polybrene and incubated for 30 min. To this mixture HIV virus 20 ng per 1*10⁷ cells was added and incubated for 2 h at 37° C. Cells were centrifuged and washed with PBS 3 times to remove unbound virus and resuspended the pellet in T75 flask with IL-2 containing 30 mL of RPMI. Incubated the cells at 37° C., 5% CO₂. Every two days' supernatant was centrifuged, and half of the media was changed with IL-2 containing RPMI. Supernatant media collected on day 7, 10 and 14 was used to estimate the amount of p24 in the culture supernatants using an ELISA kit. Aliquots of this supernatant was used for virus infection experiments.

Sequential Treatment Approach in Microglia Cells Infected with HIV-1 BaL Virus: Sequence treatments were performed to mimic HIV-infected human exposure to drugs of abuse, followed by treatment with ART-NP and NP-delivered antioxidants. Microglia cells were plated in a 6-well plate with a density of 50,000 cells per well. On day 1, cells were infected with 41 ng/mL of HIV clade-B virus and incubated for 24 h. After, the media was aspirated, and meth was added at the concentration of 500 μM and incubated for 24 h. Media was aspirated, and EFV-NP and EVG-NP were added at concentrations of 1 μM and 0.5 μM respectively with respect to the loaded cargos for 24 h. Finally, media was aspirated and T-(Asp)₄/CoQ₁₀-NPs were added at the concertation of 10 with respect to [Oc-G2-(Asp)₄] and CoQ₁₀ for additional 24 h. Each media sample was used for analysis of inflammation markers present after each stage of exposure or treatment.

Experimental approach in Microglia cells co-culture with astrocytes: Sequence treatments and co-culture of microglia cells with astrocytes were performed to mimic HIV-infected human exposure to drugs of abuse in the brain microenvironment. Microglia cells were plated in a 6-well plate with a density of 50,000 cells per well. On day 1, cells were infected with 10 μg/10,000 cells of mito-GFP for 48 h to maximize the GFP staining of the mitochondria. On day 3, cells were infected with 200 ng/mL of TAT peptide and incubated for 48 h. After that, on day 5 the media was aspirated, and 500 μM of meth was added and cells were incubated for 24 h. Media was then aspirated and the combination of T-EFV-NP+T-EVG-NP+T-DRV-NP, 0.5 μM each, were added, and cells were incubated for 24 h. On day 5 of this experiment, in a separate 6-well plate, astrocytes were plated with density of 50,000 cells per well and treated after 24 h with T-(Asp)₄-NP+T-CoQ₁₀-NP at a concentration of 10 μM each with respect to articles loaded inside. Finally, the media from the microglia culture was aspirated and the astrocytes were added and co-cultured with microglia cells for 24 h. On day 8, the microglia cells were sorted using cell sorter and tested for ROS levels using mito-SOX reagent. Each media sample was used for analysis of inflammation markers present after each stage of exposure or treatment. The experiment mentioned above was performed in live cell imaging dishes to study the Mito-SOX staining.

ATP production assay: ATP production assay was done using kit Cell Titer-Glo, Promega. 2000 cells were seeded in white wall 96 well plate and incubated overnight at 37° C. for 1 h allow the cells to settle down. The cells plate was centrifuged at 2000×g at 4° C. for 5 min. Later on the media was removed and 100 μL of Cell Titer-Glo solution was added and incubated for 10 minutes at room temperature. Luminescence signal was recorded in plate reader. The obtained values were normalized using BCA assay.

ROS production assay by Immunofluorescence: ROS production levels at mitochondria analyzed using Mito-SOX staining. On the final day of the experiment, cells were washed with PBS and mito-SOX solution (0.1 mg/mL) in DMEM was added and incubated for 30 min at room temperature in the dark. The media was removed, and the cells were then washed gently with PBS (3×). Finally, 1 mL of PBS was added to the cells and the cells were imaged using confocal microscopy. Confocal images were recorded using an Olympus FluoView FV3000 confocal microscope using 405/460 nm for DAPI and 510/580 nm for mitoSOX. Sampling speed was kept as 8.0 us/pixel (0.55 min per image). Images were analyzed using the software ImageJ.

Biodistribution of ARV-loaded NPs: The distribution and toxicity of the free ARVs and ARV-loaded nanoparticles was carried out in female BALB/c mice after intravenous administration. Saline-treated mice were used as a control. The animals were divided into seven groups: saline (n=4), EFV (n=3), DRV (n=3), EVG (n=3), T-EFV-NP (n=4), T-DRV-NP (n=4), and T-EVG-NP (n=4), here n is number of animals used in the group. The animals received saline treatment, ARV treatment or ARV-NP treatment via intravenous injection. The dose of nanoparticle was kept as 40 mg/kg with respect to ARV weight. After 24 h of injection, under anesthesia around 200 μL of blood was collected via cardiac puncture and perfusion was carried out with 1×PBS for 10 min with a flow rate of 7 mL/min. After perfusion, the animals were sacrificed, and organs were harvested. The collected blood was centrifuged to collect blood plasma. The collected organs were weighed and homogenized using a dounce homogenizer and collected in 5 mL of methanol. To spike the ARV peak, 20 μg/mL of the respective ARV was added to the crushed tissues and to the blood plasma. This mixture was sonicated for 20 min followed by centrifugation at 14000 rpm for 10 min. From the precipitated debris, supernatant was gently collected. Meanwhile, Strata-X columns were activated by passing 1 mL of methanol and 1 mL of water through the filter in sequence. The collected supernatant was passed through the activated column to get rid of remaining debris and impurities. The column was washed with 1 mL of 5% methanol to remove the impurities. The ART from the column was collected in 1.5 mL of methanol and quantified using HPLC (wavelength=efavirenz (27.3 min, 268 nm), darunavir (24.0 min, 268 nm), and elvitegravir (27.9 min, 268 nm).

Aspartate Aminotransferase (AST) Activity Assay: The collected blood plasma was used to determine AST activity. All samples and standards were studied in duplicates. AST levels in blood are commonly used as a marker for liver function. The collected serum samples were directly used to determine the AST levels. From each sample, 50 μL of serum was added to a 96-well plate. Along with this, 50 μL glutamate standards were also added, with concentrations of 0, 2, 4, 6, 8, and 10 nmol/well prepared in AST assay buffer. To each well, 100 μL of master reaction mix (80 μL of AST Assay Buffer, 2 μL of AST Enzyme Mix, 8 μL of AST Developer and 10 μL of AST Substrate) was added and incubated for 5 min at 37° C. in the dark. Meanwhile, the plate reader was set for 37° C. and kept ready to read the kinetics, with 5 min intervals, for 20 min. Absorbance at initial time, T_{Initial}, was (A₄₅₀)_{Initial}, and at the end the final time point, T_{final}, was (A₄₅₀)_{final}. The final absorbance should not exceed the highest standard (10 nmol/well) absorbance value. It is essential that (A₄₅₀) final is in the linear range of the standard curve. The absorbance was measured at 450 nm at the initial time.

Calculated the change in absorbance from Tᵢₙᵢₜᵢₐₗ to T_{final} for the samples.

$$Δ {A}_{450} = {( {A}_{450} )}_{final} - {( {A}_{450} )}_{initial}$$

The amount of generated glutamate using standard curve determined for above obtained ΔA₄₅₀ (B).

The AST activity of a sample was determined by the following equation:

$$AST Activity ( milliunit / mL ) = B / Reaction Time * V$$

B=Amount (nmole) of glutamate generated between Tᵢₙᵢₜᵢₐₗ and T_{final}. Reaction Time (min)=T_{final}−Tᵢₙᵢₜᵢₐₗ

V=sample volume (mL) added to well

Alanine Transaminase (ALT) Colorimetric Activity Assay: The collected plasma was used to determine ALT activity. Cayman's ALT Assay Kit was used to detect ALT activity in plasma. Measurement of the ALT activity is carried out by monitoring the rate of NADH oxidation in a coupled reaction system employing lactate dehydrogenase (LDH). The oxidation of NADH to NAD+ is accompanied by a decrease in absorbance at 340 nm. Under circumstances in which the ALT activity is rate limiting, the rate decrease is directly proportional to the ALT activity in the sample. This experiment was carried out in 96-well plate provided by Cayman. First, 150 μL of substrate, 20 μL of Cofactor, and 20 μL of sample were added to each well and incubated for at 37° C. for 15 min. The reaction was initiated by the addition of 20 μL of ALT initiator to all of the wells with minimal time difference between addition to the wells. Absorbance was recorded immediately at 340 nm once every minute for a period of five minutes.

The change in absorbance (ΔA340) per minute was determined by selecting two linear points on the linear portion of the curve and calculated the change in absorbance using the following equation.

$$Δ A / min = {A}_{340} ( Time 2 ) - {A}_{340} ( Time 1 ) / Time 2 ( min ) - Time 1 ( min )$$

ALT activity was determined using following formula=(ΔA₃₄₀/min×0.21 mL)×4.11 mM⁻¹×0.02 mL. The reaction rate at 340 nm can be determined using the NADH extinction coefficient of 4.11 mM⁻¹.

Creation of Mice Model of Intravenous Drug Use in HIV-Positive animals: The experiment was carried out in male C57BL/6 mice (13 weeks, Jackson laboratory) with an average weight of 25 g. C57BL/6 male mice were divided into 12 groups and were assigned to the following treatment groups: control virus+Meth+saline (12 animals); control virus+saline (12 animals); control virus+Meth+T-CoQ₁₀-NP/T-(Asp)₄-NP (13 animals); EcoHIV+Meth+saline (11 animals); EcoHIV+Meth+T-CoQ₁₀-NP/T-(Asp)₄-NP (10 animals); EcoHIV+Meth+T-ART-NPs (12 animals); EcoHIV+Meth+T-ART-NP+T-CoQ₁₀-NP/T-(Asp)₄-NP (16 animals); control Virus+Meth+T-CoQ₁₀/(Asp)₄-NPs (11 animals); control Virus+T-ART-NPs (13 animals); control Virus+T-ART-NPs+T-CoQ₁₀/(Asp)₄-NPs (12 animals), saline (3 animals), and EcoHIV+saline (3 animals). The mice were infused with a chimeric HIV-NDK (abbreviated as EcoHIV, 1 μg of p24 in 100 μl) via lateral tail vein injection.⁵ A retroviral vector pBMN-I-GFP (Addgene) was employed to generate control murine retrovirus (ConV) in Phoenix-Eco packaging cells (ATCC) (www.addgene.org/1736/). One week later those mice were injected intraperitoneally with meth three times a day with a 3 h interval. We applied an escalating dose regimen starting with 1.0 mg/kg with a constant increase of 0.2 mg/kg at each injection for 5 days. Control mice were injected with saline as a vehicle. After the last methamphetamine injection, nanoparticles were injected. T-ART-NPs were always given as a combination of the three types of drug loaded nanoparticles, T-EFV-NP, T-DRV-NP, and T-EVG-NP, and were each given at dose of 5 mg/kg with respect to each drug. T-CoQ₁₀-NP/T-(Asp)₄-NP were injected at a dose of 20 mg/kg with respect to CoQ₁₀ or (Asp)₄. The T-ART-NP followed by T-CoQ₁₀-NP/T-(Asp)₄-NP injections were conducted twice a week, for a total of 4 injections per week. Injections of T-ART-NP on day 1 would be followed by T-CoQ₁₀-NP/T-(Asp)₄-NP on day 2, and this cycle would repeat on day 4 and day 5. This entire T-ART-NP and T-CoQ₁₀-NP/T-(Asp)₄-NP treatment regimen was conducted for a total of 2 weeks. After the second week, animals were sacrificed and blood and organs were collected. Blood was collected via cardiac puncture, then the mice were perfused with PBS at 7.5 mL/min to remove trace amounts of blood from organs. The collected blood samples were centrifuged to collect blood plasma. Organs from half of the mice of each group were snap-frozen for future RT-PCR analysis, while the other half were fixed in 4% PFA for immunofluorescence and histopathology. Organs stored in PFA were submitted for sectioning for H&E analysis.

ELISA for Analyses of HIV Infection: An ELISA kit from Zeptomatrix was also used to measure levels of the p24 antigen in the blood plasma from mice. First, plates washed with washing buffer and tapped on a paper towel, after which 200 μL of standards or samples were prepared and added to the wells and the plate was incubated for 2 h at 37° C. The plate was washed with washing buffer and tapped on paper towel, and 100 μL of detector antibody was added to each well, and the plate was incubated for 1 h at 37° C. The plate was then washed again, 100 μL of substrate was added to each well, and the plate was incubated for 30 min at room temperature. Then 100 μL of stop solution was added and absorbance was recorded at 450 nm using a plate reader.

RT-PCR Study: To measure the p24 antigen RNA levels in the brain lysates of treated mice, qPCR was utilized. RNA was extracted using kit from Qiagen. Briefly, cells were lysed with buffer RLT. One volume of 70% ethanol was added to the cell lysate and mixed well. Lysates were transferred to RNeasy mini spin column and centrifuged for 1 min at 8000×g. Flow-through was discarded. 700 μL of buffer RW1 was added to the mini spin column and centrifuged for 1 for minute at 8000×g. Flow-through was discarded. A portion of 500 μL of buffer RPE was added to the mini spin column and centrifuged for 1 minute at 8000×g. RNA was recovered from mini spin column using RNase-free water. Purity and concentration of RNA was checked using Nanodrop. Reverse transcription from each sample was carried out using 1 μg of RNA from each sample in a 20 μL reaction volume using iScript Reverse Transcription Supermix for RT-qPCR kit from Bio-Rad. Real time PCR reaction was carried out using SsoAdvanced Universal SYBR© Green Supermix in 20 μL reaction. The house keeping gene, β-actin was used as an internal control. Data were analyzed according to the comparative Ct value and expressed as fold change 2-ΔΔCT. HIV was assessed using the following primers and probe: NDKgag_F 5′-GACATAAGACAGGGACCAAAGG-3′ (SEQ ID NO: 1); NDKgag_R 5′-CTGGGTTTGCATTTTGGACC-3′ (SEQ ID NO: 2). The primer sequence for β-actin gene was: Forward 5′GCATCCTCACCCTGAAGTAC 3′ (SEQ ID NO: 3) and reverse 5′GATAGCACAGCCTGGATAGC3′ (SEQ ID NO: 4). GAPDH (4326317E), C3 (Mm00437859_g1) and OLFM1 (Mm00444666_m1) was purchased from Life Technologies. The primer sequence for GCLC gene was: Forward 5′ACACCTGGATGATGCCAACGAG3′ (SEQ ID NO: 5) and reverse 5′CCTCCATTGGTCGGAACTCTAC3′ (SEQ ID NO: 6). The primer sequence for GCLM gene was: Forward 5′ TCCTGCTGTGTGATGCCACCAG3′ (SEQ ID NO: 7) and reverse 5′GCTTCCTGGAAACTTGCCTCAG3′ (SEQ ID NO: 8). The primer sequence for GPX7 gene was: Forward 5′CGACTTCAAGGCGGTCAACATC3′ (SEQ ID NO: 9) and reverse 5′AAGGCTCGGTAGTTCTGGTCTG3′ (SEQ ID NO: 10).

Immunofluorescence of Harvested Tissue Samples: Organs harvested from the treated mice were stained with antibodies for GFAP, MAP2, TMEM119, ICAM-1, catalase, and HIV-1 p24. Tissue sections were heated at 60° C. for 30 min followed by rehydration in an ethanol gradient. Antigen retrieval was carried out in a decloaking chamber. The sections were then washed with PBS (1×) 3 times and then permeabilized using 0.1% Triton-X 100 for 10 min at room temperature. The tissues were washed with 1×PBS 3 times and blocked with 1% goat serum in 1×PBS for 1 h at room temperature. Sections were treated with the respective primary antibody in 1% goat serum containing 1×PBS for 16 h at 4° C. in humidified chamber. After washing the sections three more times with 1% goat serum containing 1×PBS, the respective secondary antibody (Alexa 488 conjugated antibody) solution in 1% goat serum containing 1×PBS was added and incubated for 1 h at room temperature. Finally, DAPI stain was applied to the tissue for 5 minutes followed by 6 washes with PBS. Sections were finally washed three more times with 1% goat serum containing 1×PBS. The tissues were then covered with coverslips using mounting solution (n-propyl gallate, Tris and glycerol in nanopure water, pH=8.0). Confocal images were recorded using an Olympus FluoView FV3000 confocal microscope using 405/460 nm for DAPI and 488/510 nm for Alexa488. Sampling speed was kept as 8.0 us/pixel (0.56 min per image).

Isolation of glial cells and DCFDA assay: To isolate the glial cells and neurons from brain samples, freshly harvested brains divided into two halves, out of which one half was used for isolation of glial cells and other half for isolation of neurons. The brain samples were gently crushed using a dounce homogenizer and the suspension was collected in glial dissection media and filtered through a 100 μm cell strainer by using a sterile 30 glass tissue grinder pestle. 8 mL of glial culture medium was added drop wise to the surface of the cell strainer, and the cell suspension was collected in a 50 mL falcon tube. This cell suspension was passed through a 70 μm cell strainer, and the supernatant was collected in a new 50 mL falcon tube. The cell suspension was centrifuged at 1,000×g for 5 min and then the supernatant was aspirated. The obtained pellet was resuspended in 1 mL of glial culture medium and cells were counted, and seeded for staining and ROS activity detection on coverslips in 12 well plate and white walled, clear bottom 96-well plate respectively incubated at 37° C. with 5% CO₂ and grown for 48 h.

The remaining half of the brain was chopped into small dishes in a petri dish. These pieces were collected in neuronal dissection buffer and transferred to a 15 mL centrifuge tube. They were allowed to settle down in the buffer for 2 min, and the buffer was aspirated carefully. 1 mL trypsin was added to the pieces, and they were incubated at 37° C. in a water bath for 25 min. While incubating, the tube was agitated every 5 min. trypsin was then inactivated using pre-warmed FBS solution, and the tissue pieces were allowed to settle down at room temperature. Once the pieces settled, the supernatant was carefully aspirated. 1.5 mL of pre-warmed DNAse solution (1 mg/mL in neurobasal media) was added and incubated for 20 min at 37° C., which agitation every 5 minutes. The cells were dissociated by micropipette 20-30 times. The cells were allowed to settle at room temperature for 10 min, and the supernatant was carefully transferred to a new 15 mL centrifuge tube and centrifuged at 15,000 RPM for 5 min at 4° C. The pellet was collected in neurobasal media and counted and plated in poly-D-Lysine coated coverslips in 12 well plate and white walled clear bottom 96-well plate for immunofluorescence and ROS activity assay respectively.

ROS Activity Assay: Glial and neuronal cells plated as state above were treated with 2′,7′-dichlorofluorescein diacetate (DCFH-DA) probe with concentration of 50 μM and incubated for 30 min. The 96 well plate was centrifuged at 5000 rpm for 5 min at 4° C., the supernatant was aspirated, and cells were washed with PBS (1×) in dark condition. This centrifuge and washing step were repeated two times. After the final wash, 100 μL of PBS was added to the cells and the fluorescence was determined at 485 nm excitation and 520 nm emission, using a microplate reader.

Immunostaining of the Isolated cells: After cell isolation, cells were grown in selective media for 10 days on coverslips in a 12 well plate. The cells were washed with PBS (1×) 3 times and fixed with 4% paraformaldehyde for 15 min at 37° C. After performing 3 washings, the cells were permeabilized using 0.1% Triton-X100 for 10 min at 37° C. The cells were washed with PBS (1×) 3 times and blocked with 1% goat serum in 1×PBS for 12 h. Cells were then treated with the respective primary antibody (GFAP and NeuN antibodies) in 1% goat serum containing 1× PBS for 12 h at 4° C. in a humidified chamber. After washing the cells three times with 1% goat serum containing 1×PBS, the secondary antibody solutions in 1% goat serum containing 1×PBS were added and incubated for 1 h at room temperature in a humidified chamber. The cells were washed with 1×PBS for 3 times and DAPI (1 mg/mL in 1×PBS) was added to the cells and incubated for 5 min at room temperature. Cells were finally washed three more times with 1×PBS. The membrane was gently removed and kept on glass slides and covered with coverslips using mounting solution (n-propyl gallate, Tris and glycerol in nanopure water, pH=8.0). Confocal images were recorded using an Olympus FluoView FV3000 confocal microscope using 405/460 nm for DAPI and 488/510 nm for Alexa488.

Experimental Details for Nanoparticle Distribution Using In Vitro Placental Barrier Model: First, the transwell insert was flipped over and the HPVEC cells were plated with density of 50,000 cells per well on the top-facing basal side of the trans-well membrane in 200 μL of endothelial cell media (ECM) with 10% FBS, 1% endothelial cell growth supplement and incubated for 4 h at 37° C. in 5% CO₂. Then, the insert was gently reversed back into its original orientation, with the newly plated basal-side HPVEC cells now facing downward in 1 mL of pre-added ECM media. These cells were allowed to grow overnight. Next, on the apical side the BeWo cells were plated with the 50,000 cells per well in 500 μL of DMEM (10% FBS) media and the cells were grown up to 8 days. Before the addition of articles, the monolayer integrity was checked by measuring trans-epithelial electrical resistance (TEER) for eight days. Media was replenished once every day and TEER was measured. On the eighth day, the articles were administered on the apical side at a concentration of 20 μg/mL with respect to ARTs, and cells were incubated for 12 h. Apical basal media and the cell pellets were collected in eppendorf tubes and dissolved in 2 mL of methanol. As an internal standard, 10 μg/mL of respective ARTs were added to the collected media. This mixture was sonicated for 20 min followed by centrifugation at 5000 RPM for 10 min. From the precipitated debris, the supernatant was gently collected. Meanwhile, Strata C18-T columns were activated by passing 1 mL of methanol and water through the filter in sequence. The collected supernatant was passed through the activated column in order to get rid of remaining debris and impurities. The column was washed with 1-2 mL of 5% methanol in order to remove the impurities. The drugs from the column was collected in 2 mL of methanol and quantified using HPLC [efavirenz (27.3 min, 268 nm), darunavir (24.0 min, 268 nm), and elvitegravir (27.9 min, 268 nm)].

Biodistribution of DRV-loaded NPs in Pregnant Mice: The distribution of the targeted T-DRV-NPs was carried out in pregnant female C57BL/6 mice after intravenous administration. Female mice of 10-12 weeks were housed with male mice to induce pregnancy. On the 14^{th} day following confirmation of copulation plug, the mice were administered with NPs. Saline-treated mice were used as a control. The animals were divided into two groups: saline (n=1), T-DRV-NP (n=2). The animals received saline treatment or T-ARV-NP treatment via tail i.v. The dose of NP was kept as 40 mg/kg with respect to DRV weight. After 24 h of injection, under anesthesia, around 400 μL of blood was collected via cardiac puncture and perfusion was carried out with 1×PBS for 10 min with a flow rate of 7 mL/min. After perfusion, the animals were sacrificed and the organs were harvested. The collected blood was centrifuged to collect blood plasma. The collected organs were weighed and homogenized using a dounce homogenizer and collected in 5 mL of methanol. To spike the peak, 20 μg/mL of the DRV was added to the crushed tissues and to the blood plasma. This mixture was sonicated for 20 min followed by centrifugation at 14,000 rpm for 10 min. From the precipitated debris, supernatant was gently collected. Meanwhile, Strata-X columns were activated by passing 1 mL of methanol and 1 mL of water through the filter in sequence. The collected supernatant from the tissue was passed through the activated column to remove remaining debris and impurities. The column was washed with 1 mL of 5% methanol to remove the impurities. The ART from the column was collected in 1.5 mL of methanol and quantified using HPLC (Wavelength: 24.0 min, 268 nm).

TABLE 1
A summary of ARVs used for optimization and characterization of drug-loaded NPs.

| | | | | Diameter | Zeta Potential | | |
|---|---|---|---|---|---|---|---|
| SI. No. | ARV | Abbreviation | Activity | (nm) | (mV) | % Loading | % EE |
| 1 | Saquinavir | SQV | PI | 56 ± 1.8 | 21 ± 1.9 | 1.5 ± 2.1 | 14.8 ± 2.0 |
| 2 | Atazanavir | ATV | PI | 60 ± 2.6 | 25 ± 0.8 | ND | ND |
| 3 | Tenofovir | TDF | NRTI | 58 ± 2.3 | 22 ± 0.3 | ND | ND |
| 4 | Indinavir | IDV | PI | 56 ± 2.8 | 20 ± 1.8 | ND | ND |
| 5 | Ritonavir | RTV | PI | 58 ± 1.2 | 26 ± 1.2 | ND | ND |
| 6 | Zidovudine | ZDV | NRTI | 55 ± 1.2 | 24 ± 0.5 | ND | ND |
| 7 | Lopinavir | LPV | PI | 58 ± 1.9 | 24 ± 0.1 | ND | ND |
| 8 | Efavirenz | EFV | NNRTI | 59 ± 1.6 | 20 ± 0.9 | 4.2 ± 1.9 | 42 ± 2.0 |
| 9 | Darunavir | DRV | PI | 58 ± 1.5 | 21 ± 0.3 | 8.3 ± 2.0 | 83 ± 2.0 |
| 10 | Nevirapine | NVP | NNRTI | 55 ± 0.8 | 26 ± 0.6 | ND | ND |
| 11 | Lamivudine | 3TC | NRTI | 62 ± 1.7 | 25 ± 0.3 | ND | ND |
| 12 | Fosamprenavir | FPV | PI | 55 ± 1.7 | 23 ± 2.5 | ND | ND |
| 13 | Amprenavir | APV | PI | 57 ± 2.0 | 24 ± 2.3 | ND | ND |
| 14 | Dolutegravir | DTG | INI | 60 ± 8.5 | 24 ± 1.4 | 0.13 ± 0.8 | 1.3 ± 0.9 |
| 15 | Elvitegravir | EVG | INI | 58 ± 1.4 | 23 ± 0.6 | 1.8 ± 2.0 | 18.2 ± 2.5 |
| 16 | Cobicistat | COBI | PK Enhancer | 70 ± 2.1 | 27 ± 0.8 | ND | ND |
| 17 | Abacavir | ABC | NRTI | 57 ± 1.7 | 23 ± 4.2 | ND | ND |
| 18 | Emtricitabine | FTC | NRTI | 56 ± 1.4 | 24 ± 2.1 | ND | ND |
| 19 | Raltegravir | RAL | INI | 65 ± 1.8 | 26 ± 0.5 | 0.07 ± 0.01 | 0.8 ± 0.8 |
| 20 | Maraviroc | MVC | CCR5 Inhibitor | 65 ± 2.4 | 24 ± 0.5 | ND | ND |
| 21 | Enfuvirtide | T20 | Fusion Inhibitor | 60 ± 1.9 | 22 ± 1.4 | ND | ND |
| 22 | Delavirdine | DLV | NNRTI | 56 ± 1.4 | 19 ± 0.8 | 0.74 ± 1.0 | 7.4 ± 1.2 |
| 23 | Bictegravir | BIC | INI | 57 ± 1.4 | 29 ± 1.4 | 0.48 ± 0.9 | 4.9 ± 1.2 |
| 24 | Didanosine | ddl | NRTI | 56 ± 1.8 | 20 ± 1.3 | ND | ND |
| 25 | Zalcitabine | ddc | NRTI | 62 ± 1.7 | 22 ± 2.3 | ND | ND |
| 26 | Stavudine | d4T | NRTI | 58 ± 2.9 | 21 ± 1.4 | 0.26 ± 0.2 | 2.6 ± 1.0 |

PI: Protease Inhibitor
INI: Integrase inhibitors
NRTI: Nucleoside/Nucleotide Reverse Transcriptase Inhibitors
NNRTI: Non-nucleoside reverse transcriptase inhibitors
ND: Not detectable by HPLC

### REFERENCES

1. Fauci, A. S., The AIDS Epidemic-Considerations for the 21st Century. N. Engl. J. Med. 1999, 341, 1046-1050.
2. Piot, P.; Bartos, M.; Ghys, P. D.; Walker, N.; Schwartlander, B., The Global Impact of HIV/AIDS. Nature 2001, 410, 968-973.
3. Antinori, A.; Arendt, G.; Becker, J. T.; Brew, B. J.; Byrd, D. A.; Cherner, M.; Clifford, D. B.; Cinque, P.; Epstein, L. G.; Goodkin, K.; Gisslen, M.; Grant, I.; Heaton, R. K.; Joseph, J.; Marder, K.; Marra, C. M.; McArthur, J. C.; Nunn, M.; Price, R. W.; Sacktor, V. V., et al., Updated Research Nosology for HIV-Associated Neurocognitive Disorders. Neurology 2007, 69, 1789-1799.
4. Saylor, D.; Dickens, A. M.; Sacktor, N.; Haughey, N.; Slusher, B.; Pletnikov, M.; Mankowski, J. L.; Brown, A.; Volsky, D. J.; McArthur, J. C., HIV-Associated Neurocognitive Disorder-Pathogenesis and Prospects for Treatment. Nat. Rev. Neurol. 2016, 12, 234-248.
5. Brabers, N. A.; Nottet, H. S., Role of the Pro-Inflammatory Cytokines TNF-alpha and IL-1beta in HIV-Associated Dementia. Eur. J. Clin. Investig. 2006, 36, 447-458.
6. Akay, C.; Lindl, K. A.; Shyam, N.; Nabet, B.; Goenaga-Vazquez, Y.; Ruzbarsky, J.; Wang, Y.; Kolson, D. L.; Jordan-Sciutto, K. L., Activation Status of Integrated Stress Response Pathways in Neurones and Astrocytes of HIV-Associated Neurocognitive Disorders (HAND) Cortex. Neuropathol. Appl. Neurobiol. 2012, 38, 175-200.
7. Dutta, R.; Roy, S., Mechanism(s) Involved in Opioid Drug Abuse Modulation of HAND. Curr. HIV Res. 2012, 10, 469-477.
8. Sanchez, A. B.; Kaul, M., Neuronal Stress and Injury Caused by HIV-1, cART and Drug Abuse: Converging Contributions to HAND. Brain Sci. 2017, 7, 1-22.
9. Hazleton, J. E.; Berman, J. W.; Eugenin, E. A., Novel Mechanisms of Central Nervous System Damage in HIV Infection. HIV AIDS (Auckl.) 2010, 2, 39-49.
10. Lindl, K. A.; Marks, D. R.; Kolson, D. L.; Jordan-Sciutto, K. L., HIV-Associated Neurocognitive Disorder: Pathogenesis and Therapeutic Opportunities. J. Neuroimmune Pharmacol. 2010, 5, 294-309.
11. Ton, H.; Xiong, H., Astrocyte Dysfunctions and HIV-1 Neurotoxicity. J. AIDS Clin. Res. 2013, 4, 255-267.
12. Zayyad, Z.; Spudich, S., Neuropathogenesis of HIV: From Initial Neuroinvasion to HIV-Associated Neurocognitive Disorder (HAND). Curr. HIV/AIDS Rep. 2015, 12, 16-24.
13. Kaul, M.; Garden, G. A.; Lipton, S. A., Pathways to Neuronal Injury and Apoptosis in HIV-Associated Dementia. Nature 2001, 410, 988-994.
14. Surnar, B.; Basu, U.; Banik, B.; Ahmad, A.; Marples, B.; Kolishetti, N.; Dhar, S., Nanotechnology-Mediated Crossing of Two Impermeable Membranes to Modulate the Stars of the Neurovascular Unit for Neuroprotection. Proc. Natl. Acad. Sci. U.S. A. 2018, 115, E12333-E12342.
15. Velichkovska, M.; Surnar, B.; Nair, M.; Dhar, S.; Toborek, M., Targeted Mitochondrial CoQ10 Delivery Attenuates Antiretroviral Drug-Induced Senescence of Neural Progenitor Cells. Mol. Pharm. 2018, 16, 724-736.
16. Kalathil, A. A.; Kumar, A.; Banik, B.; Ruiter, T. A.; Pathak, R. K.; Dhar, S., New Formulation of Old Aspirin for Better Delivery. Chem. Commun. 2016, 52, 140-143.
17. Feldhaeusser, B.; Platt, S. R.; Marrache, S.; Kolishetti, N.; Pathak, R. K.; Montgomery, D. J.; Reno, L. R.; Howerth, E.; Dhar, S., Evaluation of Nanoparticle Delivered Cisplatin in Beagles. Nanoscale 2015, 7, 13822-13830.
18. Marrache, S.; Pathak, R. K.; Dhar, S., Detouring of Cisplatin to Access Mitochondrial Genome for Overcoming Resistance Proc. Natl. Acad. Sci. U.S.A 2014, 111, 10444-10449.
19. Marrache, S.; Tundup, S.; Harn, D. A.; Dhar, S., Ex Vivo Programming of Dendritic Cells by Mitochondria-Targeted Nanoparticles to Produce Interferon-Gamma for Cancer Immunotherapy. ACS Nano 2013, 7, 7392-7402.
20. Hung, S. W.; Mody, H.; Marrache, S.; Bhutia, Y. D.; Davis, F.; Cho, J. H.; Zastre, J.; Dhar, S.; Chu, C. K.; Govindarajan, R., Pharmacological Reversal of Histone Methylation Presensitizes Pancreatic Cancer Cells to Nucleoside Drugs: In Vitro Optimization and Novel Nanoparticle Delivery Studies. PLoS One 2013, 8, e71196-e71199.
21. Marrache, S.; Dhar, S., Engineering of Blended Nanoparticle Platform for Delivery of Mitochondria-Acting Therapeutics. Proc. Natl. Acad. Sci. U.S.A 2012, 109, 16288-16293.
22. Chen, Y.; Swanson, R. A., Astrocytes and Brain Injury. J. Cereb. Blood Flow Metab. 2003, 23, 137-149.
23. Voloboueva, L. A.; Suh, S. W.; Swanson, R. A.; Giffard, R. G., Inhibition of Mitochondrial Function in Astrocytes: Implications for Neuroprotection. J. Neurochem. 2007, 102, 1383-1394.
24. Samikkannu, T.; Rao, K. V.; Kanthikeel, S. P.; Atluri, V. S.; Agudelo, M.; Roy, U.; Nair, M. P., Immunoneuropathogenesis of HIV-1 Clades B and C: Role of Redox Expression and Thiol Modification. Free Radic. Biol. Med. 2014, 69, 136-144.
25. Geraghty, P.; Hadas, E.; Kim, B.-H.; Dabo, A. J.; Volsky, D. J.; Foronjy, R., HIV Infection Model of Chronic Obstructive Pulmonary Disease in Mice Am. J. Physiol. Lung Cell Mol. 2017, 312, L500-L509.
26. Gu, C.-J.; Borjabad, A.; Hadas, E.; Kelschenbach, J.; Kim, B.-H.; Chao, W.; Arancio, O.; Suh, J.; Polsky, B.; McMillan, J.; Edagwa, B.; Gendelman, H. E.; Potash, M. J.; Volsky, D. J., EcoHIV Infection of Mice Establishes Latent Viral Reservoirs in T Cells and Active Viral Reservoirs in Macrophages that are Sufficient for Induction of Neurocognitive Impairment. PLoS Pathog. 2018, 14, e1007061-e1007061.
27. Jones, L. D.; Jackson, J. W.; Maggirwar, S. B., Modeling HIV-1 Induced Neuroinflammation in Mice: Role of Platelets in Mediating Blood-Brain Barrier Dysfunction. PLoS One 2016, 11, e0151702-e0151709.
28. Spire, B.; Sire, J.; Zachar, V.; Rey, F.; Barre-Sinoussi, F.; Galibert, F.; Hampe, A.; Chermann, J.-C., Nucleotide Sequence of HIV1-NDK: a Highly Cytopathic Strain of the Human Immunodeficiency Virus. Gene 1989, 81, 275-284.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### SEQUENCES

(SEQ ID NO: 1)
5′-GACATAAGACAGGGACCAAAGG-3′
(SEQ ID NO: 2)
5′-CTGGGTTTGCATTTTGGACC-3′
(SEQ ID NO: 3)
5′GCATCCTCACCCTGAAGTAC3′
(SEQ ID NO: 4)
5′GATAGCACAGCCTGGATAGC3′
(SEQ ID NO: 5)
5′ACACCTGGATGATGCCAACGAG3′
(SEQ ID NO: 6)
5′CCTCCATTGGTCGGAACTCTAC3′
(SEQ ID NO: 7)
5′TCCTGCTGTGTGATGCCACCAG3′
(SEQ ID NO: 8)
5′GCTTCCTGGAAACTTGCCTCAG3′
(SEQ ID NO: 9)
5′CGACTTCAAGGCGGTCAACATC3′
(SEQ ID NO: 10)
5′AAGGCTCGGTAGTTCTGGTCTG3′

1. A method for reducing a level of human immunodeficiency virus (HIV) in a pregnant subject in need thereof, comprising administering to the pregnant subject a therapeutically effective amount of the nanoparticle, wherein the nanoparticle comprises:
a mitochondrial targeting moiety;
an anti-HIV therapeutic agent; and/or
one or more of an anti-oxidant agent and/or an anti-inflammatory agent.

2. The method of claim 1, wherein the nanoparticle comprises the anti-oxidant agent or the anti-inflammatory agent.

3. The method of claim 1, wherein the anti-oxidant comprises Coenzyme Q10 (CoQ₁₀).

4. The method of claim 1, wherein the anti-inflammatory agent comprises a prodrug of aspirin or a prodrug of prednisone.

5. The method of claim 4, wherein the prodrug of aspirin is Oc-G2-(Asp)₄ (Asp4).

6. The method of claim 4, wherein the prodrug of prednisone is Oc-[G-2]-(Pred)₄.

7. The method of claim 1, wherein the anti-HIV therapeutic agent comprises a protease inhibitor, an integrase inhibitor, a fusion inhibitor, a nucleoside reverse transcriptase inhibitor (NRTI), a nucleotide reverse transcriptase inhibitor (NtRTI), a non-nucleoside reverse-transcriptase inhibitor (NNRTI), a CCR5 antagonist, a post attachment inhibitor, or a maturation inhibitor.

8. The method of claim 7, wherein the integrase inhibitor comprises raltegravir, elvitegravir, dolutegravir, bictegravir, V-165, BI 224436, MK-2048, Lens epithelial derived growth factor LEDGF, or cabotegravir.

9. The method of claim 7, wherein the protease inhibitor comprises amprenavir, lopinavir Kaletra, saquinavir, indinavir, ritonavir, nelfinavir, atazanavir, fosamprenavir, tipranavir, darunavir, DMP450, PNU-140690, ABT-378, PD178390, asunaprevir, boceprevir, grazoprevir, glecaprevir, paritaprevir, simeprevir, telaprevir, tanomastat, batimastat, nelfinavir, or bortezomib.

10. The method of claim 7, wherein the NRTI comprises zidovudine, didanosine, didanosine EC, zalcitabine, stavudine, lamivudine, abacavir, emtricitabine, entecavir, or truvada.

11. The method of claim 7, wherein the NtRTI comprises tenofovir or adefovir.

12. The method of claim 7, wherein the NNRTI comprises efavirenz, nevirapine, delavirdine, etravirine, rilpivirine, etravirine, or doravirine.

13. The method of claim 7, wherein the CCR5 antagonist comprises leronlimab, aplaviroc, vicriviroc, maraviroc, or INCB009471.

14. The method of claim 7, wherein the fusion inhibitor comprises efuvirtide.

15. The method of claim 7, wherein the anti-HIV therapeutic agent comprises saquinavir, darunavir, elvitegravir, dolutegravir, raltegravir, bictegravir, efavirenz, delavirdine, remdesivir, or stavudine.

16. The method of claim 6, wherein the anti-HIV therapeutic agent comprises saquinavir, efavirenz, or elvitegravir.

17. The method of claim 1, wherein the nanoparticle comprises poly(lactic-co-glycolic acid) (PLGA)-block(b)-polyethylene glycol (PEG).

18. The method of claim 1, wherein the mitochondrial targeting moiety comprises a triphenyl phosphonium (TPP) moiety or a derivative thereof.

19. The method of claim 1, wherein the nanoparticle has a diameter of about 100 nm or less.

20.-24. (canceled)

25. A method for treating human immunodeficiency virus (HIV)-associated neurocognitive disorder (HAND) in a pregnant subject in need thereof, comprising administering to the pregnant subject a therapeutically effective amount of the nanoparticle, wherein the nanoparticle comprises:
a mitochondrial targeting moiety;
an anti-HIV therapeutic agent; and/or
one or more of an anti-oxidant agent and/or an anti-inflammatory agent.


SEQUENCE LISTING (10 sequences)

SEQ ID NO: 1 - DNA, 22 bp, synthetic construct
  source 1..22: mol_type = other DNA
gacataagacagggaccaaagg

SEQ ID NO: 2 - DNA, 20 bp, synthetic construct
  source 1..20: mol_type = other DNA
ctgggtttgcattttggacc

SEQ ID NO: 3 - DNA, 20 bp, synthetic construct
  source 1..20: mol_type = other DNA
gcatcctcaccctgaagtac

SEQ ID NO: 4 - DNA, 20 bp, synthetic construct
  source 1..20: mol_type = other DNA
gatagcacagcctggatagc

SEQ ID NO: 5 - DNA, 22 bp, synthetic construct
  source 1..22: mol_type = other DNA
acacctggatgatgccaacgag

SEQ ID NO: 6 - DNA, 22 bp, synthetic construct
  source 1..22: mol_type = other DNA
cctccattggtcggaactctac

SEQ ID NO: 7 - DNA, 22 bp, synthetic construct
  source 1..22: mol_type = other DNA
tcctgctgtgtgatgccaccag

SEQ ID NO: 8 - DNA, 22 bp, synthetic construct
  source 1..22: mol_type = other DNA
gcttcctggaaacttgcctcag

SEQ ID NO: 9 - DNA, 22 bp, synthetic construct
  source 1..22: mol_type = other DNA
cgacttcaaggcggtcaacatc

SEQ ID NO: 10 - DNA, 22 bp, synthetic construct
  source 1..22: mol_type = other DNA
aaggctcggtagttctggtctg